(19) United States
(12) Patent Application Publication
(10) Pub. No.: US 2022/0195516 A1
(43) Pub. Date: Jun. 23, 2022

(54) METHODS, SYSTEMS AND COMPOSITIONS FOR NUCLEIC ACID SEQUENCING

(71) Applicant: Illumina Cambridge Limited, Cambridge (GB)

(72) Inventors: Xiaohai LIU, Cambridge (GB); Xiaolin WU, Cambridge (GB); Patrick MCCAULEY, Cambridge (GB); Nikolai Nikolaevich ROMANOV, Cambridge (GB); Elena CRESSINA, Cambridge (GB); Antoine FRANCAIS, Cambridge (GB)

(21) Appl. No.: 17/550,262

(22) Filed: Dec. 14, 2021

Related U.S. Application Data
(60) Provisional application No. 63/127,043, filed on Dec. 17, 2020.

(51) Int. Cl. C12Q 1/6874; G01N 21/64

(52) U.S. Cl. CPC C12Q 1/6874; G01N 2021/6419; G01N 21/6428

(57) ABSTRACT

The present disclosure relates to methods, systems, kits and compositions for nucleic acid sequencing applications. In particular, the method utilizes two imaging events with different excitation wavelengths and a single emission channel to collect the fluorescent signal patterns of different types of nucleotide conjugates to determine the identity of the incorporated nucleotide conjugates. The method described herein does not require a chemical treatment of the nucleotide conjugates in the incorporation mixture between the two imaging events.

### INCORPORATION BY REFERENCE TO ANY PRIORITY APPLICATION

The present application claims the benefit of priority to U.S. Ser. No. 63/127,043, filed Dec. 17, 2020, which is incorporated by reference in its entirety.

### FIELD

The present disclosure generally relates to methods, systems, kits and compositions for nucleic acid sequencing applications.

### BACKGROUND

Nucleic acid sequencing methodology has evolved significantly from the chemical degradation methods used by Maxam and Gilbert and the strand elongation methods used by Sanger. Today several sequencing methodologies are in use which allow for the parallel processing of thousands of nucleic acids all in a single sequencing run. The instrumentation that performs such methods is typically large and expensive since the current methods typically rely on large amounts of expensive reagents and multiple sets of optic filters to record nucleic acid incorporation into sequencing reactions.

It has become clear that the need for high-throughput, smaller, less expensive DNA sequencing technologies will be beneficial for reaping the rewards of genome sequencing. Personalized healthcare is moving toward the forefront and will benefit from such technologies. The sequencing of an individual's genome to identify potential mutations and abnormalities will be crucial in identifying if a person has a particular disease, followed by subsequent therapies tailored to that individual. To accommodate such endeavor, sequencing technologies should not only have high throughput capabilities, but also have scalability. As such, there exist a need for new sequencing methods that with improvement on speed, error read, and are also cost effective.

### SUMMARY

The present disclosure provides next-generation sequencing methods, systems and compositions. Some embodiments of the present disclosure relate to a method for determining the sequence of a target polynucleotide, comprising:

(a) contacting a primer polynucleotide with a mixture (i.e., incorporation mixture) comprising one or more of four different types of nucleotide conjugates, wherein a first type of nucleotide conjugate comprises a first label, a second type of nucleotide conjugate comprises a second label, and a third type of nucleotide conjugate comprises a third label, wherein each of the first label, the second label, and the third label is spectrally distinct from one another, and wherein the primer polynucleotide is complementary to at least a portion of the target polynucleotide;

(b) incorporating a nucleotide conjugate from the mixture in the primer polynucleotide to produce an extended primer polynucleotide;

(c) performing a first imaging event using a first excitation light source and collecting a first emission signal from the extended primer polynucleotide; and

(d) performing a second imaging event using a second excitation light source and collecting a second emission signal from the extended primer polynucleotide;

wherein the first excitation light source and the second excitation light source have different wavelengths; and wherein first emission signal and the second emission signal are detected or collected in a single emission detection channel/filter. In some embodiments, the method does not comprise a chemical modification of any nucleotide conjugates in the mixture after the first imaging event and prior to the second imaging event. In some embodiments, the mixture further comprises a fourth type of nucleotide conjugate, wherein the fourth type of nucleotide conjugate is either unlabeled or is labeled with a fluorescent moiety that does not emit a signal from either the first or the second imaging event. In some further embodiments, each of the one or more of four different types of nucleotide conjugates in the incorporation mixture contains a 3′ hydroxyl blocking group.

In the sequencing method described herein, the identity of each incorporated nucleotide conjugate is determined based on the detection patterns of the first imaging event and the second imaging event. For example, the incorporation of the first type of the nucleotide conjugate is determined by a signal state in the first imaging event and a dark state in the second imaging event. The incorporation of the second type of the nucleotide conjugates is determined by a dark state in the first imaging event and a signal state in the second imaging event. The incorporation of the third type of the nucleotide conjugates is determined by a signal state in both the first imaging event and the second imaging event. The incorporation of the fourth type of the nucleotide conjugates is determined by a dark state in both the first imaging event and the second imaging event. In some embodiments, the mixture in step (a) comprises four different types of nucleotide conjugates (A, C, G, and T or U). In some further embodiments, three of the four types of nucleotide conjugates are each labeled with a fluorophore that is spectrally distinct from another, and one of the nucleotides is not labeled with a fluorophore. In further embodiments, steps (a) through (d) are performed in repeated cycles (e.g., at least 30, 50, 100, 150, 200, 250 or 300 times) and the method further comprises sequentially determining the identity of each incorporated nucleotide conjugates, thereby determining the sequence of at least a portion of the single-stranded polynucleotide.

Additional embodiments of the present disclosure relate to a kit for sequencing application, comprising:

a first type of nucleotide conjugate comprises a first label;

a second type of nucleotide conjugate comprises a second label; and

a third type of nucleotide conjugate comprises a third label;

wherein each of the first label, the second label, and the third label is spectrally distinct from one another, the first label and the third label are excitable using a first light source wavelength, the second label and the third label are excitable using a second light source wavelength that is different from the first light source wavelength; and wherein each of the first label, the second label and the third label has an emission spectrum that is detectable in a single detection channel. In some embodiments, the kit comprises four different types of nucleotide conjugates (A, C, G, and T or U). In some further embodiments, three of the four types of nucleotide conjugates are each labeled with a fluorophore that is spectrally distinct from another, and one of the nucleotides is not labeled with a fluorophore.

### BRIEF DESCRIPTION OF THE DRAWINGS

FIG. 1A illustrates a flowchart for the standard Illumina one-channel sequencing-by-synthesis (SBS) chemistry.

FIG. 1B illustrates how Image 1 and Image 2 from standard one-channel SBS are processed by image analysis software to identify which bases are incorporated.

FIG. 2 illustrates an embodiment of the sequencing method described herein.

FIG. 3A illustrates an embodiment of a detection system for the sequencing method described herein.

FIG. 3B illustrates an embodiment of the single emission detection channel described herein.

FIG. 4 illustrates a scatterplot obtained on an iSeq™ 100 system according to an embodiment of the sequencing method described herein

### DETAILED DESCRIPTION

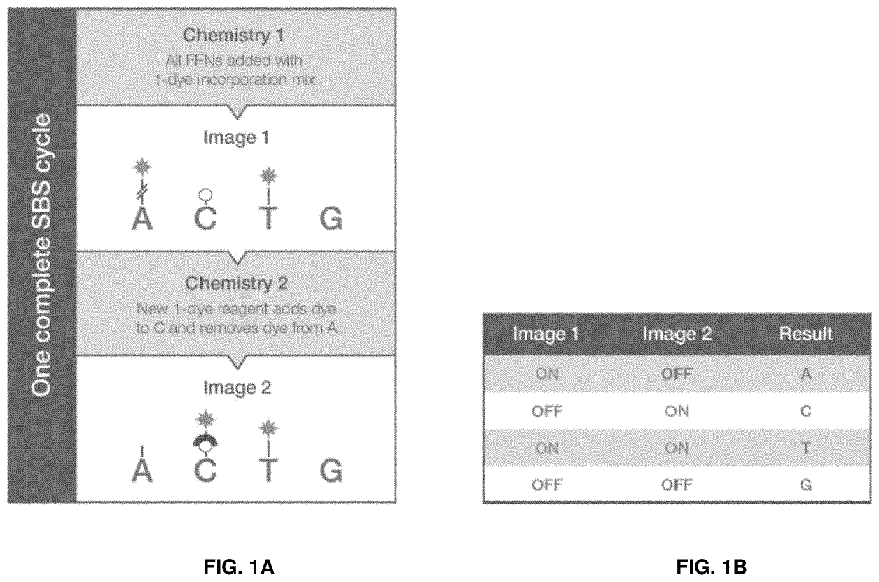

Illumina's Next-Generation Sequencing system, the iSeq™ 100 uses a complementary metal-oxide-semiconductor (CMOS)-based technology to deliver a simplified, accessible benchtop sequencing solution. The standard sequencing workflow is illustrated in FIGS. 1A and 1B, which is also referred to as the one-channel sequencing. Each sequencing cycle includes two chemistry steps and two imaging steps. In FIG. 1A, the first chemistry step exposes the flowcell to a mixture of nucleotides that have fluorescently labeled adenines and thymines. During the first imaging step, the light emission from each cluster is recorded by the CMOS sensor. The second chemistry step removes the fluorescent label from adenine and adds a fluorescent label to cytosine. In both chemistry steps, guanine is dark (unlabeled). The second image is recorded. In FIG. 1B, the combination of Image 1 and Image 2 are processed by image analysis software to identify which bases are incorporated at each cluster position. This sequencing cycle is repeated “n” times to create a read length of “n” bases. Unlike four-channel SBS chemistry, where sequencers use a different dye for each nucleotide, the iSeq™ 100 System uses one dye per sequencing cycle. In one-channel chemistry, adenine has a removable label and is labeled in the first image only. Cytosine has a linker group that can bind a label and is labeled in the second image only. Thymine has a permanent fluorescent label and is therefore labeled in both images, and guanine is permanently dark. Nucleotides are identified by analysis of the different emission patterns for each base across the two images.

The present disclosure provides alternative and improved solutions to the one channel sequencing described in Illumina's the iSeq™ 100 platform. In particular, the present disclosure provides methods for determining the identity of the incorporation of a nucleotide using two imaging steps and one emission detection filter/channel. The use of fewer filter allows for sequencing to be performed on smaller footprint. Furthermore, the methods of the present disclosure eliminate the second chemical step described above. The methods and systems as described herein decrease instrument hardware needs, decrease the size of an instrument, reagent usage and costs while increasing data output speed and accuracy. For example, the method described herein may be used on Illumina's iSeq™ sequencing system, providing fast turnaround times and efficient sequencing.

### Definitions

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps but may include additional steps. When used in the context of a compound, composition, or device, the term “comprising” means that the compound, composition, or device includes at least the recited features or components, but may also include additional features or components.

As used herein, common organic abbreviations are defined as follows:
° C. Temperature in degrees Centigrade
dATP Deoxyadenosine triphosphate
dCTP Deoxycytidine triphosphate
dGTP Deoxyguanosine triphosphate
dTTP Deoxythymidine triphosphate
ddNTP Dideoxynucleotide triphosphate
ffA Fully functionalized A nucleotide
ffC Fully functionalized C nucleotide
ffG Fully functionalized G nucleotide
ffN Fully functionalized nucleotide
ffT Fully functionalized T nucleotide
LED Light emitting diode
SBS Sequencing by synthesis

As used herein, the term “array” refers to a population of different probe molecules that are attached to one or more substrates such that the different probe molecules can be differentiated from each other according to relative location. An array can include different probe molecules that are each located at a different addressable location on a substrate. Alternatively, or additionally, an array can include separate substrates each bearing a different probe molecule, wherein the different probe molecules can be identified according to the locations of the substrates on a surface to which the substrates are attached or according to the locations of the substrates in a liquid. Exemplary arrays in which separate substrates are located on a surface include, without limitation, those including beads in wells as described, for example, in U.S. Pat. No. 6,355,431 B1, US 2002/0102578 and PCT Publication No. WO 00/63437. Exemplary formats that can be used in the invention to distinguish beads in a liquid array, for example, using a microfluidic device, such as a fluorescent activated cell sorter (FACS), are described, for example, in U.S. Pat. No. 6,524,793. Further examples of arrays that can be used in the invention include, without limitation, those described in U.S. Pat. Nos. 5,429,807; 5,436,327; 5,561,071; 5,583,211; 5,658,734; 5,837,858; 5,874,219; 5,919,523; 6,136,269; 6,287,768; 6,287,776; 6,288,220; 6,297,006; 6,291,193; 6,346,413; 6,416,949; 6,482,591; 6,514,751 and 6,610,482; and WO 93/17126; WO 95/11995; WO 95/35505; EP 742 287; and EP 799 897.

As used herein, the term “covalently attached” or “covalently bonded” refers to the forming of a chemical bonding that is characterized by the sharing of pairs of electrons between atoms. For example, a covalently attached polymer coating refers to a polymer coating that forms chemical bonds with a functionalized surface of a substrate, as compared to attachment to the surface via other means, for example, adhesion or electrostatic interaction. It will be appreciated that polymers that are attached covalently to a surface can also be bonded via means in addition to covalent attachment.

In each instance where a single mesomeric form of a compound described herein is shown, the alternative mesomeric forms are equally contemplated.

As used herein, a “nucleotide” includes a nitrogen containing heterocyclic base, a sugar, and one or more phosphate groups. They are monomeric units of a nucleic acid sequence. In RNA, the sugar is a ribose, and in DNA a deoxyribose, i.e. a sugar lacking a hydroxyl group that is present in ribose. The nitrogen containing heterocyclic base can be purine, deazapurine, or pyrimidine base. Purine bases include adenine (A) and guanine (G), and modified derivatives or analogs thereof, such as 7-deaza adenine or 7-deaza guanine. Pyrimidine bases include cytosine (C), thymine (T), and uracil (U), and modified derivatives or analogs thereof. The C-1 atom of deoxyribose is bonded to N-1 of a pyrimidine or N-9 of a purine.

As used herein, a “nucleotide conjugate” generally refers to a nucleotide labeled with a fluorescent moiety, optionally through a cleavage linker as described herein. In some embodiment, when a nucleotide conjugate is described as an unlabeled nucleotide conjugate, such nucleotide does not include a fluorescent moiety. In some further embodiments, an unlabeled nucleotide conjugate also does not have a cleavable linker.

As used herein, a “nucleoside” is structurally similar to a nucleotide but is missing the phosphate moieties. An example of a nucleoside analogue would be one in which the label is linked to the base and there is no phosphate group attached to the sugar molecule. The term “nucleoside” is used herein in its ordinary sense as understood by those skilled in the art. Examples include, but are not limited to, a ribonucleoside comprising a ribose moiety and a deoxyribonucleoside comprising a deoxyribose moiety. A modified pentose moiety is a pentose moiety in which an oxygen atom has been replaced with a carbon and/or a carbon has been replaced with a sulfur or an oxygen atom. A “nucleoside” is a monomer that can have a substituted base and/or sugar moiety. Additionally, a nucleoside can be incorporated into larger DNA and/or RNA polymers and oligomers.

The term “purine base” is used herein in its ordinary sense as understood by those skilled in the art and includes its tautomers. Similarly, the term “pyrimidine base” is used herein in its ordinary sense as understood by those skilled in the art and includes its tautomers. A non-limiting list of optionally substituted purine-bases includes purine, adenine, guanine, deazapurine, 7-deaza adenine, 7-deaza guanine. hypoxanthine, xanthine, alloxanthine, 7-alkylguanine (e.g., 7-methylguanine), theobromine, caffeine, uric acid and isoguanine. Examples of pyrimidine bases include, but are not limited to, cytosine, thymine, uracil, 5,6-dihydrouracil and 5-alkylcytosine (e.g., 5-methylcytosine).

As used herein, when an oligonucleotide or polynucleotide is described as “comprising” a nucleoside or nucleotide described herein, it means that the nucleoside or nucleotide described herein forms a covalent bond with the oligonucleotide or polynucleotide. Similarly, when a nucleoside or nucleotide is described as part of an oligonucleotide or polynucleotide, such as “incorporated into” an oligonucleotide or polynucleotide, it means that the nucleoside or nucleotide described herein forms a covalent bond with the oligonucleotide or polynucleotide. In some such embodiments, the covalent bond is formed between a 3′ hydroxy group of the oligonucleotide or polynucleotide with the 5′ phosphate group of a nucleotide described herein as a phosphodiester bond between the 3′ carbon atom of the oligonucleotide or polynucleotide and the 5′ carbon atom of the nucleotide.

As used herein, the term “cleavable linker” is not meant to imply that the whole linker is required to be removed. The cleavage site can be located at a position on the linker that ensures that part of the linker remains attached to the detectable label and/or nucleoside or nucleotide moiety after cleavage.

As used herein, “derivative” or “analog” means a synthetic nucleotide or nucleoside derivative having modified base moieties and/or modified sugar moieties. Such derivatives and analogs are discussed in, e.g., Scheit, Nucleotide Analogs (John Wiley & Son, 1980) and Uhlman et al., Chemical Reviews 90:543-584, 1990. Nucleotide analogs can also comprise modified phosphodiester linkages, including phosphorothioate, phosphorodithioate, alkyl-phosphonate, phosphoranilidate and phosphoramidate linkages. “Derivative”, “analog” and “modified” as used herein, may be used interchangeably, and are encompassed by the terms “nucleotide” and “nucleoside” defined herein.

As used herein, the term “phosphate” is used in its ordinary sense as understood by those skilled in the art, and includes its protonated forms (for example,

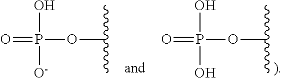 [SMILES: *OP(=O)([O-])O ; *OP(=O)(O)O]

As used herein, the terms “monophosphate,” “diphosphate,” and “triphosphate” are used in their ordinary sense as understood by those skilled in the art and include protonated forms.

As understood by one of ordinary skill in the art, a compound such as a nucleotide conjugate described herein may exist in ionized form, e.g., containing a —CO₂⁻, —SO₃⁻ or —O⁻. If a compound contains a positively or negatively charged substituent group, it may also contain a negatively or positively charged counterion such that the compound as a whole is neutral. In other aspects, the compound may exist in a salt form, where the counterion is provided by a conjugate acid or base.

As used herein, the term “phasing” refers to a phenomenon in SBS that is caused by incomplete removal of the 3′ terminators and fluorophores, and/or failure to complete the incorporation of a portion of DNA strands within clusters by polymerases at a given sequencing cycle. Prephasing is caused by the incorporation of nucleotides without effective 3′ terminators, wherein the incorporation event goes 1 cycle ahead due to a termination failure. Phasing and prephasing cause the measured signal intensities for a specific cycle to consist of the signal from the current cycle as well as noise from the preceding and following cycles. As the number of cycles increases, the fraction of sequences per cluster affected by phasing and prephasing increases, hampering the identification of the correct base. Prephasing can be caused by the presence of a trace amount of unprotected or unblocked 3′-OH nucleotides during sequencing by synthesis (SBS). The unprotected 3′-OH nucleotides could be generated during the manufacturing processes or possibly during the storage and reagent handling processes.

### Sequencing Methods

Some aspect of the present disclosure relates to a method for determining the sequence of a target polynucleotide, e.g., a single-stranded target polynucleotide, comprising:

(a) contacting a primer polynucleotide with a mixture comprising one or more of four different types of nucleotide conjugates, wherein a first type of nucleotide conjugate comprises a first label, a second type of nucleotide conjugate comprises a second label, and a third type of nucleotide conjugate comprises a third label, wherein each of the first label, the second label, and the third label is spectrally distinct from one another, and wherein the primer polynucleotide is complementary to at least a portion of the target polynucleotide;

(b) incorporating a nucleotide conjugate from the mixture in the primer polynucleotide to produce an extended primer polynucleotide;

(c) performing a first imaging event using a first excitation light source and detecting/collecting a first emission signal from the extended primer polynucleotide;

(d) performing a second imaging event using a second excitation light source and detecting/collecting a second emission signal from the extended primer polynucleotide;

wherein the first excitation light source and the second excitation light source have different wavelengths; and wherein first emission signal and the second emission signal are detected or collected in a single emission detection channel.

In some embodiments of the sequencing method described herein, the incorporation mixture further comprises a fourth type of nucleotide, wherein the fourth type of nucleotide is unlabeled of is labeled with a fluorescent moiety that does not emit a detectable signal from either the first or the second imaging event. In some embodiments of the method, the first emission signal comprises signal from the first label of the first type of nucleotide conjugate or the third label of the third type of nucleotide conjugate. In some embodiments of the method, the second emission signal comprises signal from the second label of the second type of nucleotide conjugate or the third label of the third type of nucleotide conjugate. In some embodiments, the incorporation of the first type of the nucleotide conjugate is determined by a signal state in the first imaging event and a dark state in the second imaging event. In some embodiments, the incorporation of the second type of the nucleotide conjugates is determined by a dark state in the first imaging event and a signal state in the second imaging event. In some embodiments, the incorporation of the third type of the nucleotide conjugates is determined by a signal state in both the first imaging event and the second imaging event. In some embodiments, the incorporation of the fourth type of the nucleotide conjugates is determined by a dark state in both the first imaging event and the second imaging event. In one embodiment, the incorporation of the first type of the nucleotide conjugates is determined by a signal state in the first imaging event and a dark state in the second imaging event; the incorporation of the second type of the nucleotide conjugates is determined by a dark state in the first imaging event and a signal state in the second imaging event; the incorporation of the third type of the nucleotide conjugates is determined by a signal state in the first imaging event and second imaging event; and the incorporation of a fourth type of the nucleotide conjugates is determined by a dark state in the first imaging event and second imaging event.

The term “signal state” when used in reference to an imaging event, refers to the state of a labeled nucleotide conjugate, in which a specific emission signal is produced by such imaging event, and the emission signal is detected or collected in a single detection channel/filter described herein (i.e., one channel detection). For example, a fluorescent moiety in a nucleotide conjugate may be excited by a light source (e.g., a laser) at a specific wavelength and emits a fluorescent signal that is collected or detected in the single emission detection channel/filter, indicating a “signal state” in such imaging event.

The term “dark state,” when used in reference to an imaging event, refers to the state of a labeled nucleotide conjugate, in which either no specific emission signal is produced by such imaging event, or no emission signal is collected or detected in the single emission detection channel/filter. A “dark state” of a nucleotide conjugate may result from various situations. In one scenario, such nucleotide conjugate lacks a fluorescent moiety and as a result, it does not emit any fluorescent signals. In another scenario, the nucleotide conjugate is labeled with a fluorescent moiety that cannot be excited by a light source at a specific wavelength and therefore cannot emit any signal or emits minimal fluorescence (e.g., a red emission dye may not be excitable at a wavelength in the blue or violet region). In a third scenario, the nucleotide conjugate is labeled with a fluorescent moiety, and emits a signal as a result of the imaging event. However, the wavelength of such emission signal is outside the single detection channel and thus cannot be detected (e.g., a dye emits a blue signal, but the detection channel is in the green to red region). Dark state detection may also include any background fluorescence which may be present absent a fluorescent label. For example, some reaction components may demonstrate minimal fluorescence when excited at certain wavelengths. As such, even though there is not a fluorescent moiety present there may be background fluorescence from such components. Further, background fluorescence may be due to light scatter, for example from adjacent sequencing reactions, which may be detected by a detector. In addition, background fluorescence may be caused by impure clusters (e.g., due to multiple template seeding during cluster amplification, phasing or prephasing events). As such, “dark state” can include such background fluorescence as when a fluorescent moiety is not specifically included, such as when a nucleotide lacking a fluorescent label is utilized in methods described herein. However, such background fluorescence is contemplated to be differentiable from a signal state and as such nucleotide incorporation of an unlabeled nucleotide (or “dark” nucleotide) is still discernible.

In one example of the method described herein, “T” nucleotide conjugate is determined by a signal state in the first imaging event and a dark state in the second imaging event; “C” nucleotide conjugate is determined by a dark state in the first imaging event and a signal state in the second imaging event; “A” nucleotide conjugate is determined by a signal state in the first imaging event and a signal state in the second imaging event; and “G” nucleotide conjugate is determined by a dark state in the first imaging event and a dark state in the second imaging event. In another example, “C” nucleotide conjugate is determined by a signal state in the first imaging event and a dark state in the second imaging event; “T” nucleotide conjugate is determined by a dark state in the first imaging event and a signal state in the second imaging event; “A” nucleotide conjugate is determined by a signal state in the first imaging event and a signal state in the second imaging event; and “G” nucleotide conjugate is determined by a dark state in the first imaging event and a dark state in the second imaging event. Non-limiting examples are illustrated in Table A below. In each example, “T” may also be replaced by “U”.

TABLE A
Identity of nucleotide conjugate based on first and second imaging event

| Example | First Imaging Event | Second Imaging Event | Result |
|---|---|---|---|
| 1 | Signal state | Dark state | C |
| | Dark state | Signal state | T |
| | Signal state | Signal state | A |
| | Dark state | Dark state | G |
| 2 | Signal state | Dark state | T |
| | Dark state | Signal state | C |
| | Signal state | Signal state | A |
| | Dark state | Dark state | G |
| 3 | Signal state | Dark state | A |
| | Dark state | Signal state | C |
| | Signal state | Signal state | T |
| | Dark state | Dark state | G |
| 4 | Signal state | Dark state | C |
| | Dark state | Signal state | A |
| | Signal state | Signal state | T |
| | Dark state | Dark state | G |
| 5 | Signal state | Dark state | A |
| | Dark state | Signal state | T |
| | Signal state | Signal state | C |
| | Dark state | Dark state | G |
| 6 | Signal state | Dark state | T |
| | Dark state | Signal state | A |
| | Signal state | Signal state | C |
| | Dark state | Dark state | G |
| 7 | Signal state | Dark state | G |
| | Dark state | Signal state | T |
| | Signal state | Signal state | A |
| | Dark state | Dark state | C |
| 8 | Signal state | Dark state | T |
| | Dark state | Signal state | G |
| | Signal state | Signal state | A |
| | Dark state | Dark state | C |
| 9 | Signal state | Dark state | A |
| | Dark state | Signal state | T |
| | Signal state | Signal state | G |
| | Dark state | Dark state | C |
| 10 | Signal state | Dark state | T |
| | Dark state | Signal state | A |
| | Signal state | Signal state | G |
| | Dark state | Dark state | C |
| 11 | Signal state | Dark state | G |
| | Dark state | Signal state | A |
| | Signal state | Signal state | T |
| | Dark state | Dark state | C |
| 12 | Signal state | Dark state | A |
| | Dark state | Signal state | G |
| | Signal state | Signal state | T |
| | Dark state | Dark state | C |

In some embodiments of the method described herein, the nucleotide conjugates in the mixture in step (a) comprise nucleotide types selected from the group consisting of A, C, G, T and U, and non-natural nucleotide analogs thereof. In further embodiments, the mixture in step (a) comprises four different types of nucleotide conjugates (A, C, G, and T or U), or non-natural nucleotide analogs thereof. In further embodiments, the four different types of nucleotide conjugates are dATP, dCTP, dGTP and dTTP or dUTP, or non-natural nucleotide analogs thereof. In some further embodiments, three of the four types of nucleotide conjugates are each labeled with a fluorophore that is spectrally distinct from another, and one of the nucleotide conjugates is not labeled with a fluorophore, or is labeled with a fluorophore but cannot be exited and emits a signal in either the first imaging or the second imaging event. In further embodiments, each of the four types of nucleotide conjugates in the incorporation mixture contains a 3′ hydroxyl blocking group.

In some embodiments of the method described herein, the method further includes step (e): cleaving or removing the fluorescent label from the incorporated nucleotide conjugate prior to the next sequencing cycle (e.g., after the second imaging event, and prior to the start of the next sequencing cycle. In further embodiments, the incorporated nucleotide conjugate has a 3′ hydroxy blocking group and the 3′ hydroxy blocking group is also removed prior to the next sequencing cycle. In some such embodiments, the label and the 3′ hydroxy blocking group are removed in a single step (e.g., under the same chemical reaction condition). In other embodiments, the label and the 3′ hydroxy blocking group are removed in two separate steps (e.g., the label and the 3′ blocking group are removed under two separate chemical reaction conditions). In some further embodiments, a post cleavage washing step is used after the label and the 3′ blocking group are removed. In further embodiments, steps (a) through (e) are performed in repeated cycles (e.g., at least 30, 50, 100, 150, 200, 250, 300, 400, or 500 times) and the method further comprises sequentially determining the sequence of at least a portion of the target polynucleotide based on the identity of each sequentially incorporated nucleotide conjugates. In some such embodiments, steps (a) through (e) are repeated at least 50 cycles. In some such embodiments, the four different types of nucleotide conjugates are simultaneously present and compete for incorporation during each cycle. In some further embodiments, the incorporation of the nucleotide conjugates is performed by a polymerase (e.g., a DNA polymerase). Exemplary polymerases include but not limited to Pol 812, Pol 1901, Pol 1558 or Pol 963. The amino acid sequences of Pol 812, Pol 1901, Pol 1558 or Pol 963 DNA polymerases are described, for example, in U.S. Patent Publication Nos. 2020/0131484 A1 and 2020/0181587 A1, both of which are incorporated by references herein.

In some embodiments of the method described herein, each of the first excitation light source used in the first imaging event and the second excitation light source used in the second imaging event comprises a laser, a light-emitting diode (LED), or a combination thereof. In some embodiments, the first excitation light source has a shorter wavelength than the second excitation light source. In some such embodiments, the first excitation light source has a wavelength of about 400 nm to about 480 nm, about 420 nm to about 470 nm, or about 450 nm to about 460 nm (i.e., “blue light”). In one embodiment, the first excitation light source has a wavelength of about 450 nm. The second excitation light source has a wavelength of about 490 nm to about 550 nm, about 500 nm to about 540 nm, or about 510 nm to about 530 nm (i.e., “green light”). In one embodiment, the second excitation light source has a wavelength of about 520 nm. In other embodiments, the first excitation light source has a longer wavelength than the second excitation light source. In some such embodiments, the first excitation light source has a wavelength of about 490 nm to about 550 nm, about 500 nm to about 540 nm, or about 510 nm to about 530 nm (i.e., “green light”). In one embodiment, the second excitation light source has a wavelength of about 520 nm. The second excitation light source has a wavelength of about 400 nm to about 480 nm, about 420 nm to about 470 nm, or about 450 nm to about 460 nm (i.e., “blue light”). In one embodiment, the second excitation light source has a wavelength of about 450 nm.

In some embodiments of the method described herein, the single emission detection channel has a detection spectrum range above about 560 nm, above about 570 nm, above about 580 nm, above about 590 nm, or above about 600 nm. In further embodiments, the single emission detection has a detection spectrum range that is less than about 700 nm, less than about 690 nm, less than about 680 nm, less than about 670 nm, less than about 660 nm, or less than about 650 nm. In some further embodiments, the single emission detection channel has a detection spectrum range from about 560 nm to about 690 nm, from about 570 nm to about 680 nm, or from about 580 nm to about 650 nm. The term “a single emission detection channel” as used herein, refers to a detection channel or a filter that only allows light of certain region of the spectra or within certain wavelength range to be detected, while any emission outside such spectra region will not be detected. In particular, each of the first label, the second label, and the third label has an emission spectrum that overlaps at a certain wavelength range such that the emission from any of the first label, the second label and the third label can be detected by the same detection channel or filter. In some such embodiments, the emission spectrum that can be detected by the single emission detection channel from about 560 nm to about 690 nm, from about 570 nm to about 680 nm, or from about 580 nm to about 650 nm.

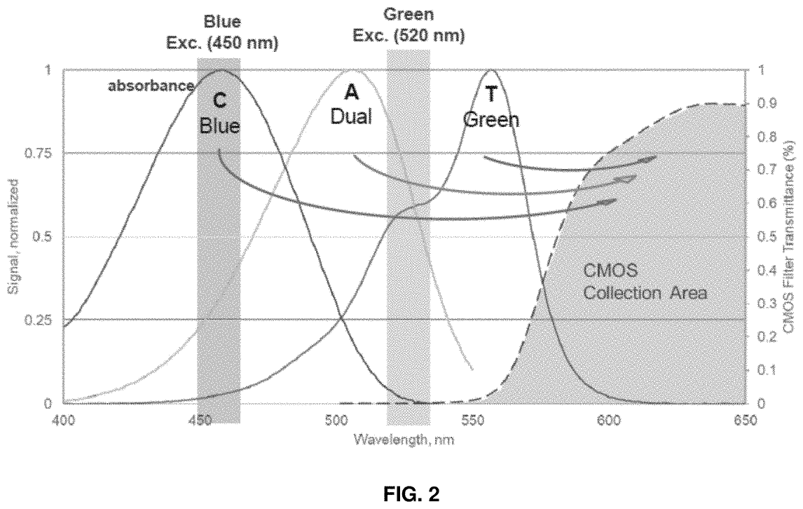

FIG. 2 illustrates an embodiment of the imaging events described in the sequencing method. The incorporation mixture contains four nucleotides: a dCTP labeled with a first dye (ffC) that is excitable by a blue light source with wavelength at about 450 nm; a dTTP labeled with a second dye (ffT) that is excitable by a green light source with wavelength at about 520 nm; a dATP labeled with a third dye (ffA) that is excitable by both the blue light source at 450 nm and the green light source at 540 nm (“dual dye”); and dGTP (ffG) which is unlabeled. The emission spectra of the ffC, ffT and ffA are detected in the CMOS collection area that is above 560 nm (e.g., from about 570 nm to about 650 nm). When the first imaging event uses the blue light source and the second imaging event uses the green light source, the identity of the incorporated nucleotide conjugates can be determined as the following:

| Identity of nucleotide | First Imaging Event | Second Imaging Event |
|---|---|---|
| C | Signal state | Dark state |
| T | Dark state | Signal state |
| A | Signal state | Signal state |
| G | Dark state | Dark state |

Similarly, when the first imaging event uses the green light source and the second imaging event uses the blue light source, the identity of the incorporated nucleotide conjugates can be determined as the following:

| Identity of nucleotide | First Imaging Event | Second Imaging Event |
|---|---|---|
| T | Signal state | Dark state |
| C | Dark state | Signal state |
| A | Signal state | Signal state |
| G | Dark state | Dark state |

The two-excitation, single channel detection sequencing method described herein require that the fluorescent dyes not only have strong fluorescent and chemically stable, but also have tailor-made absorption and long Stokes shifts. In some embodiments, the nucleotide conjugate with the dual dye that is excitable at both the first and the second light source (e.g., 450 nm and 520 nm, or 520 nm and 450 nm) has an absorbance maximum (Aₘₐₓ) of about 480 nm to 510 nm, or about 490 nm to 500 nm. The nucleotide conjugate with the dye that is excitable only at a shorter wavelength (either the first light source or the second light source) has an Aₘₐₓ of about 450-460 nm. The nucleotide conjugate with the dye that is excitable only at a longer wavelength (either the first light source or the second light source) has an Aₘₐₓ of over 520 nm. In further embodiments, the emission maximum (Eₘₐₓ) of the nucleotide conjugate with the dual dye is greater than 550 nm or greater than 560 nm. The Stokes shift of such labeled nucleotide conjugate is greater than 60 nm. In some further embodiments, the Eₘₐₓ of the nucleotide conjugate with the dye that is excitable only at a shorter wavelength (shown signal state by shorter wavelength light source and dark state by longer wavelength light source) is greater than 560 nm. The Stokes shift of such labeled nucleotide conjugate is greater than 100 nm. In some further embodiments, the Eₘₐₓ of the nucleotide conjugate with the dye that is excitable only at a longer wavelength is greater than 560 nm. The Stokes shift of such nucleotide conjugate is greater than about 30 nm or greater than about 40 nm. Non-limiting examples of coumarin dyes with Aₘₐₓ between 450-460 nm, 480-510 nm or 490-500 nm that may be used in the sequencing method described herein include those disclosed in U.S. Publication Nos. 2018/0094140 A1, 2018/0201981 A1, 2020/0277529 A1 and 2020/0277670 A1 and U.S. Ser. No. 63/057,758, which are incorporated by references. Non-limiting examples of polymethine dyes with Aₘₐₓ greater than 520 nm that may be used in the sequencing method described herein include those disclosed in International Patent Publication Nos. WO 2013/041117, WO 2014/135221, WO 2015/170102, WO 2016/189287 and WO 2017/051201.

In some embodiments, the combination of emission detection from the first imaging event and the second imaging event are processed by image analysis software to determine the identity of the bases are incorporated at each immobilized primer polynucleotide/target polynucleotide complex position. In some such embodiments, the image analysis is processed after repeated cycles of incorporation (after at least 50, 100, 150, 200, 250 or 300 runs). In further embodiments, multiple immobilized primer polynucleotide/target polynucleotide complexes (clusters) are detected and sequenced in parallel.

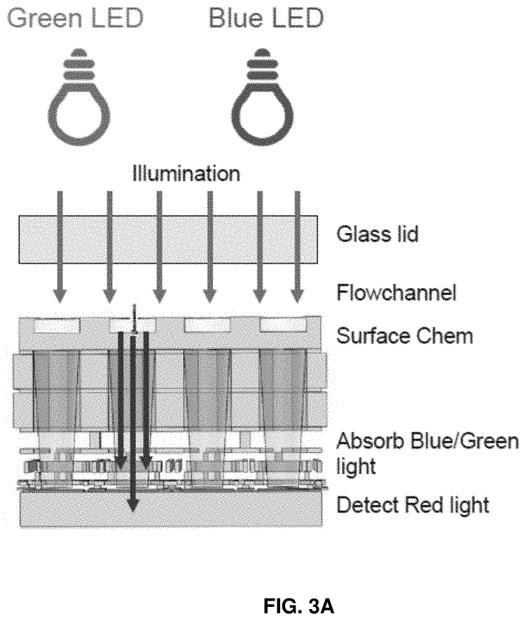

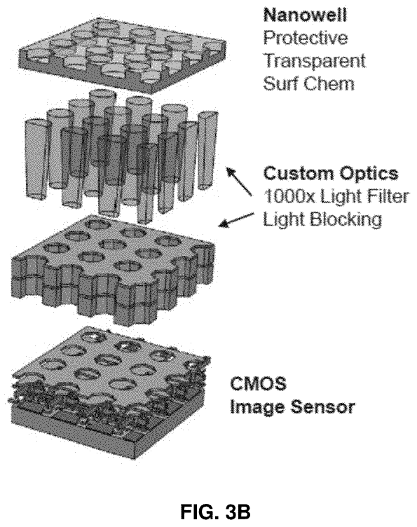

FIG. 3A illustrates a perspective view of a system used for a single channel detection. The solid support (e.g., a flow cell) contains clustered target polynucleotide templates immobilized on the surface, and primer polynucleotides hybridized to at least a portion of the templates, forming clustered primer polynucleotide/target polynucleotide complexes. The flow channel includes discrete nanowells, each containing an immobilized primer polynucleotide/target polynucleotide complex, where an incorporated nucleotide conjugate is exposed to green light source and blue light source excitation respectively. Depending on the fluorescent moiety attached to the incorporated nucleotide conjugate, it may absorb either one of the blue/green light, both the blue/green light, or neither the blue/green light (e.g., when the incorporated nucleotide conjugate does not contain a fluorescent moiety). The emission of the fluorescent moiety (e.g., red light) is detected in a single detection channel). The workflow is simplified solely for illustrating the imaging events and the detection mechanism. This workflow does not include additional steps of the sequencing method described herein, for example, the cleaving of the fluorescent moiety after the second imaging event, and optional post cleavage washing step.

FIG. 3B is a perspective view of an exemplary sequencing and detection mechanism 300 used in the sequencing method described herein. 300 may be integrated into a solid support described herein. First of all, a patterned flowcell 310 comprises a plurality of discrete nanowells 315, separated by interstitial regions. The sequencing reactions (e.g., nucleotide incorporation, deprotection of the 3′ blocking of the incorporated nucleotide conjugate, cleaving the label after detection) are performed within each of the nanowells. The flowcell may also contain a protective surface that is transparent, allowing the blue light and green light illumination to go through. 320 is a plurality of optic filters that are embedded into a light blocking element 325 such that only the emission signal generated from the incorporated nucleotide conjugate that is in a specific wavelength range can pass through and be detected by the CMOS image sensor.

In any embodiments of the methods described herein, the target polynucleotide, e.g., a single-stranded target polynucleotide, may be immobilized to a solid support. In some such embodiments, the solid support comprises a plurality of immobilized target polynucleotides, e.g., single-stranded target polynucleotides. In further embodiments, each target polynucleotide is hybridized to a primer polynucleotide that is complementary to at least a portion of the target polynucleotide and therefore forming a primer nucleotide/target nucleotide complex. The solid support may comprise clustered primer nucleotide/target nucleotide complexes. In some embodiments, the solid support comprises a flowcell, for example, a patterned flowcell comprising a plurality of nanowells, each separate from one another. In some further embodiments, each nanowell comprises one immobilized cluster therein. In some embodiment, the solid support further comprises a CMOS chip. For example, the patterned flow cell is fabricated on top of the CMOS chip, separated by a plurality of optic filters and a light blocking elements such that the emission signal generated from the imaging event is filtered through the optics filters and detected by the COMS image sensors. In still further embodiments, each of the plurality of nanowells are aligned directly over each CMOS photodiode (pixel).

In any embodiments of the method described herein, the method does not comprise any chemical modification of any nucleotide conjugates in the mixture after the first imaging event and prior to the second imaging event. The chemical modification may utilize chemical reagents such as cleavage reagents, binding partner-fluorescent moiety conjugates, or other reagents that may directly or indirectly cause an identifiable and measurable change in fluorescence from the first imaging event to the second imaging event. For example, the chemical modification may include cleaving the label from one or more types of nucleotide conjugate and/or adding a label to one or more different types of nucleotide conjugate. In particular, the method described herein eliminates the second chemistry step required for the iSeq™ 100 sequencer, which removes the fluorescent label from one nucleotide conjugate and adds a fluorescent label to a different type of nucleotide conjugate. As a result, the sequencing method described herein provides a substantial reduction of cycle time and reduces the volume of the required reagents (e.g., THP reagent, and washing solutions). In some embodiments, the sequencing method described herein provides at least 5%, 10%, 15%, 20%, 25%, or 30% reduction in cycle time compared to standard cycle time on Illumina's iSeq™ 100 using the one dye, one detection method described in FIG. 1A.

Additional illustrative embodiments are described below.

In some embodiments, methods for sequencing a nucleic acid comprise the use of one fluorescent labels for direct or indirect detection of three different nucleotide types and one nucleotide type that is not detected by the presence of a fluorescent signal but is instead detected by a lack or absence of a fluorescent signal. In some embodiments, methods for sequencing a nucleic acid comprise the use of two or more different fluorescent labels that comprise the same or similar excitation/emission spectra for direct or indirect detection of three different nucleotide types and one nucleotide type that is not detected by the presence of a fluorescent signal but is instead detected by a lack or absence of fluorescent signal. The same or similar excitation and emission spectra are such that a light source excites the two or more different fluorescent labels, and an optical filter captures their emitted fluorescence signals. Detection of fluorescence to determine the sequence of a nucleic acid sample is performed in time space, for example at different times during a sequencing reaction (i.e., pre and post a change in reaction conditions such as enzymatic cleavage, change in environmental pH, addition of additional reagents), providing patterns of fluorescence such as fluorescence transitions patterns, their cumulative patterns determining the sequence of the nucleic acid target. As such, the methods described herein are time and cost efficient and allow for simplification of associated sequencing instrumentation.

An exemplary application of utilizing time space fluorescence pattern differences for determining a target nucleic acid sequence is sequence by synthesis (SBS) methodologies and technologies. As such, embodiments as described herein find particular utility in sequence by synthesis fluorescent applications. Even though embodiments as described herein are exemplary of innovative methods of fluorescent sequencing, the disclosed embodiments also find utility for a variety of other applications where detection of more than one analyte (i.e., nucleotide, protein, or fragments thereof) in a sample is desired.

In some embodiments, the sequencing is performed on a substrate, such as a glass, plastic, semiconductor chip or composite derived substrate. In some embodiments, one nucleic acid species is provided on a substrate for example for single target sequencing. In other embodiments, sequencing can also be in a multiplex format, wherein multiple nucleic acid targets are detected and sequenced in parallel, for example in a flowcell or array type of format. Embodiments described herein are particularly advantageous when practicing parallel sequencing or massive parallel sequencing. Platforms practicing fluorescent parallel sequencing include, but are not limited to, those offered by Illumina, Inc. (e.g., HiSeq®, Genome Analyzer, MiSeq™, MiniSeq™, NextSeq™, iSeq™, and NovaSeq™ platforms), Life Technologies (e.g., SOLiD), Helicos Biosciences (e.g., Heliscope), 454/Roche Life Sciences (Branford, Conn.) and Pacific Biosciences (e.g., SMART). Flowcells, chips, and other types of surfaces that may accommodate multiple nucleic acid species are exemplary of substrates utilized for parallel sequencing. In multiplex formats wherein multiple nucleic acid species are sequenced in parallel, clonally amplified target sequences (e.g., via emulsion PCR (emPCR) or bridge amplification) are typically covalently immobilized on a substrate. For example, when practicing emulsion PCR, the target of interest is immobilized on a bead, whereas clonally amplified targets are immobilized in channels of a flowcell or specific locations on an array or chip.

Flowcells for use with compositions and methods as described herein can be used in sequencing in a number of ways. For example, a DNA sample such as a DNA library can be applied to a flowcell or fluidic device comprising one or more etched flow channels, wherein the flowcell can further comprise a population of probe molecules covalently attached to its surface. The probes attached in the flowcell channels are advantageously located at different addressable locations in the channel and DNA library molecules can be added to the flowcell channels wherein complementary sequences can bind (as described herein, further as described in WO 2012/096703, which is incorporated herein by reference in its entirety). Another example of a flowcell for use in the present application comprises a CMOS flowcell as described in U.S. Pat. Nos. 8,906,320 and 9,990,381 which is incorporated herein by reference in its entirety. Bridge amplification can be performed as described herein followed by sequencing by synthesis methods and compositions as described herein. Methods for creating and utilizing flowcells for sequencing are known in the art; references to which are provided herein and all of which are incorporated herein by reference in their entireties. It is contemplated that the methods and compositions as described herein are not limited to any particular manufacture or method of flowcell directed sequencing methodologies.

Sequencing utilizing the methods and compositions described herein can also be performed in a microtiter plate, for example in high density reaction plates or slides (Margulies et al., 2005, Nature 437(7057): 376-380, incorporated herein by reference in its entirety). For example, genomic targets can be prepared by emPCR technologies. Reaction plates or slides can be created from fiber optic material capable of capturing and recording light generated from a reaction, for example from a fluorescent or luminescent reaction. The core material can be etched to provide discrete reaction wells capable of holding at least one emPCR reaction bead. Such slides/plates can contain over a 1.6 million wells. The created slides/plates can be loaded with the target sequencing reaction emPCR beads and mounted to an instrument where the sequencing reagents are provided, and sequencing occurs.

An example of arrayed substrates for sequencing targets utilizing compositions and methods as disclosed herein is provided when practicing patterned substrates comprising DNA nanoballs on a chip or slide as performed by Complete Genomics (Mountain View, Calif.). As described in Drmanac et al., 2010, Science 327(5961): 78-81, a silicon wafer can be layered with silicon dioxide and titanium and subsequently patterned using photolithography and dry etching techniques. The wafer can be treated with HMDS and coated with a photoresist layer to define discrete regions for silanization and subsequent covalent attachment of DNA nanoballs for sequencing. A skilled artisan will appreciate that many methods exist for creating slides/chips with discrete locations for immobilization of nucleic acids for use in sequencing methodologies and the present methods are not limited by the method in which a substrate is prepared for sequencing.

For purposes of illustration and not intended to limit embodiments as described herein, a general strategy sequencing cycle can be described by a sequence of steps. The following example is based on a sequence by synthesis sequencing reaction, however the methods as described herein as not limited to any particular sequencing reaction methodology.

Alternatively, the sequencing method described herein may also be carried out using unlabeled nucleotides while employing the same two imaging events and single channel emission detection described above. For example, one, two, three or each of the four different types of nucleotides (e.g., dATP, dCTP, dGTP and dTTP or dUTP) in the incorporation mixture of step (a) may be unlabeled. Each of the four types of nucleotides (e.g., dNTPs) has a 3′ hydroxyl blocking group to ensure that only a single base can be added by a polymerase to the 3′ end of the primer polynucleotide. After incorporation of an unlabeled nucleotide in step (b), the remaining unincorporated nucleotides are washed away. An affinity reagent is then introduced that specifically recognizes and binds to the incorporated dNTP to provide a labeled extension product comprising the incorporated dNTP. Uses of unlabeled nucleotides and affinity reagents in sequencing by synthesis have been disclosed in WO 2018/129214 and WO 2020/097607. A modified sequencing method of the present disclosure using unlabeled nucleotides may include the following steps:

(a′) contacting a primer polynucleotide with a mixture comprising one or more of four different types of unlabeled nucleotides, wherein the primer polynucleotide is complementary to at least a portion of the single stranded target polynucleotide;

(b′) incorporating one unlabeled nucleotide from the mixture in the primer polynucleotide to produce an extended primer polynucleotide;

(c′) contacting the extended primer polynucleotide with a set of different affinity reagents under conditions wherein one affinity reagent binds specifically to the incorporated unlabeled nucleotide to provide a labeled extended primer polynucleotide;

(d′) performing a first imaging event using a first excitation light source and detecting a first emission signal from the labeled extended primer polynucleotide;

(e′) performing a second imaging event using a second excitation light source and detecting a second emission signal from the labeled extended primer polynucleotide;

wherein the first excitation light source and the second excitation light source have different wavelengths; and wherein first emission signal and the second emission signal are detected or collected in a single emission filter or channel. In some embodiments of the modified sequencing method described herein, each of the unlabeled nucleotides in the incorporation mixture contains a 3′ hydroxyl blocking group. In further embodiments, the 3′ hydroxyl blocking group of the incorporated nucleotide is removed prior to the next sequencing cycle. In still further embodiments, the method further comprises removing the affinity reagent from the incorporated nucleotide. In still further embodiments, the 3′ hydroxyl blocking group and the affinity reagent are removed in the same reaction. In some embodiments, the set of affinity reagents may comprise a first affinity reagent that binds specifically to the first type of nucleotide, a second affinity reagent that binds specifically to the second type of nucleotide, and a third affinity reagent that binds specifically to the third type of nucleotide. In some further embodiments, each of the first, second and the third affinity reagents comprises a detectable labeled. In some embodiments, the affinity reagents include protein tags, antibodies (including but not limited to binding fragments of antibodies, single chain antibodies, bispecific antibodies, and the like), aptamers, knottins, affimers, or any other known agent that binds an incorporated nucleotide with a suitable specificity and affinity. In one embodiment, the affinity reagent is a protein tag or an antibody. In another embodiment, the affinity reagent is a protein tag or an antibody comprising one or more detectable labels that is a fluorescent label.

The four nucleotide types A, C, T and G, typically modified nucleotides designed for sequencing reactions having a 3′ hydroxyl blocking group wherein three of the four types are fluorescently labelled, are simultaneously added, along with other reaction components, to a location where the template sequence of interest (also used interchangeably as a target polynucleotide sequence of interest) is located and the sequencing reaction occurs (e.g., flowcell, chip, slide, etc.). Following incorporation of a nucleotide into a growing sequence nucleic acid chain based on the target sequence, the reaction is exposed to light and any emission signal is captured and recorded; this constitutes a first imaging event and a first fluorescence detection pattern. Following the first imaging event, the sample is irradiated with a second light having a different wavelength from the first light source, and the reaction location is once again illuminated and any change in fluorescence is captured and recorded; constituting a second imaging event (i.e., a second fluorescence detection pattern). 3′ blocking group on the incorporated nucleotides are removed and washed away along with other reagents present after the second imaging event in preparation for the next sequencing cycle. In some embodiments, the method of the present disclosure does not involve the use of any chemical reagents that may directly cause an identifiable and measurable change in fluorescence from the first imaging event to the second imaging event. The fluorescence patterns from the two imaging events are compared and nucleotide incorporation, and thus the sequence of the target nucleic acid, for that particular cycle is determined.

In one embodiment, at least one nucleotide is incorporated into a polynucleotide (such as a single stranded primer polynucleotide described herein) in the synthetic step by the action of a polymerase enzyme. However, other methods of joining nucleotides to polynucleotides, such as, for example, chemical oligonucleotide synthesis or ligation of labeled oligonucleotides to unlabeled oligonucleotides, can be used. Therefore, the term “incorporating,” when used in reference to a nucleotide and polynucleotide, can encompass polynucleotide synthesis by chemical methods as well as enzymatic methods.

In a specific embodiment, a synthetic step is carried out and may optionally comprise incubating a template or target polynucleotide strand with a reaction mixture comprising fluorescently labeled nucleotides of the disclosure. A polymerase can also be provided under conditions which permit formation of a phosphodiester linkage between a free 3′ OH group on a polynucleotide strand annealed to the template or target polynucleotide strand and a 5′ phosphate group on the labeled nucleotide. Thus, a synthetic step can include formation of a polynucleotide strand as directed by complementary base-pairing of nucleotides to a template/target strand.

In all embodiments of the methods, the detection step may be carried out while the polynucleotide strand into which the labeled nucleotides are incorporated is annealed to a target strand, or after a denaturation step in which the two strands are separated. Further steps, for example chemical or enzymatic reaction steps or purification steps, may be included between the synthetic step and the detection step. In particular, the polynucleotide strand incorporating the labeled nucleotide(s) may be isolated or purified and then processed further or used in a subsequent analysis. By way of example, polynucleotide strand incorporating the labeled nucleotide(s) as described herein in a synthetic step may be subsequently used as labeled probes or primers. In other embodiments, the product of the synthetic step set forth herein may be subject to further reaction steps and, if desired, the product of these subsequent steps purified or isolated.

Suitable conditions for the synthetic step will be well known to those familiar with standard molecular biology techniques. In one embodiment, a synthetic step may be analogous to a standard primer extension reaction using nucleotide precursors, including the labeled nucleotides as described herein, to form an extended polynucleotide strand (primer polynucleotide strand) complementary to the target strand in the presence of a suitable polymerase enzyme. In other embodiments, the synthetic step may itself form part of an amplification reaction producing a labeled double stranded amplification product comprised of annealed complementary strands derived from copying of the primer target polynucleotide strands. Other exemplary synthetic steps include nick translation, strand displacement polymerization, random primed DNA labeling, etc. A particularly useful polymerase enzyme for a synthetic step is one that is capable of catalyzing the incorporation of the labeled nucleotides as set forth herein. A variety of naturally occurring or mutant/modified polymerases can be used. By way of example, a thermostable polymerase can be used for a synthetic reaction that is carried out using thermocycling conditions, whereas a thermostable polymerase may not be desired for isothermal primer extension reactions. Suitable thermostable polymerases which are capable of incorporating the labeled nucleotides according to the disclosure include those described in WO 2005/024010 or WO06120433, each of which is incorporated herein by reference. In synthetic reactions which are carried out at lower temperatures such as 37° C., polymerase enzymes need not necessarily be thermostable polymerases, therefore the choice of polymerase will depend on a number of factors such as reaction temperature, pH, strand-displacing activity and the like.

In specific non-limiting embodiments, the disclosure encompasses methods of nucleic acid sequencing, re-sequencing, whole genome sequencing, single nucleotide polymorphism scoring, any other application involving the detection of the modified nucleotide or nucleoside labeled with dyes set forth herein when incorporated into a polynucleotide.

In a particular embodiment the disclosure provides use of labeled nucleotides comprising dye moiety according to the disclosure in a polynucleotide sequencing-by-synthesis reaction. Sequencing-by-synthesis generally involves sequential addition of one or more nucleotides or oligonucleotides to a growing polynucleotide chain in the 5′ to 3′ direction using a polymerase or ligase in order to form an extended polynucleotide chain complementary to the template/target nucleic acid to be sequenced. The identity of the base present in one or more of the added nucleotides can be determined in a detection or “imaging” step. The identity of the added base may be determined after each nucleotide incorporation step. The sequence of the template may then be inferred using conventional Watson-Crick base-pairing rules. The use of the nucleotides labeled with dyes set forth herein for determination of the identity of a single base may be useful, for example, in the scoring of single nucleotide polymorphisms, and such single base extension reactions are within the scope of this disclosure.

In an embodiment of the present disclosure, the sequence of a target polynucleotide is determined by detecting the incorporation of one or more nucleotides into a nascent strand complementary to the target polynucleotide to be sequenced through the detection of fluorescent label(s) attached to the incorporated nucleotide(s). Sequencing of the target polynucleotide can be primed with a suitable primer (or prepared as a hairpin construct which will contain the primer as part of the hairpin), and the nascent chain is extended in a stepwise manner by addition of nucleotides to the 3′ end of the primer in a polymerase-catalyzed reaction.

In particular embodiments, each of the different nucleotide triphosphates (A, T, G and C) may be labeled with a unique fluorophore and also comprises a blocking group at the 3′ position to prevent uncontrolled polymerization. Alternatively, one of the four nucleotides may be unlabeled (dark). The polymerase enzyme incorporates a nucleotide into the nascent chain complementary to the template/target polynucleotide, and the blocking group prevents further incorporation of nucleotides. Any unincorporated nucleotides can be washed away and the fluorescent signal pattern from each incorporated nucleotide can be “read” optically by suitable means, such as a charge-coupled device using excitation and suitable emission filters. The 3′ blocking group and fluorescent dye compounds can then be removed (cleaved) (simultaneously or sequentially) to expose the nascent chain for further nucleotide incorporation. Typically, the identity of the incorporated nucleotide will be determined after each incorporation step, but this is not strictly essential. Similarly, U.S. Pat. No. 5,302,509 (which is incorporated herein by reference) discloses a method to sequence polynucleotides immobilized on a solid support.

The method, as exemplified above, utilizes the incorporation of fluorescently labeled, 3′ blocked nucleotides A, G, C, and T into a growing strand complementary to the immobilized polynucleotide, in the presence of DNA polymerase. The polymerase incorporates a base complementary to the target polynucleotide but is prevented from further addition by the 3′ hydroxyl blocking group. The label of the incorporated nucleotide can then be determined, and the blocking group removed by chemical cleavage to allow further polymerization to occur. The nucleic acid template to be sequenced in a sequencing-by-synthesis reaction may be any polynucleotide that it is desired to sequence. The nucleic acid template for a sequencing reaction will typically comprise a double stranded region having a free 3′ OH group that serves as a primer or initiation point for the addition of further nucleotides in the sequencing reaction. The region of the template to be sequenced will overhang this free 3′ OH group on the complementary strand. The overhanging region of the template to be sequenced may be single stranded but can be double-stranded, provided that a “nick is present” on the strand complementary to the target strand to be sequenced to provide a free 3′ OH group for initiation of the sequencing reaction. In such embodiments, sequencing may proceed by strand displacement. In certain embodiments, a primer bearing the free 3′ OH group may be added as a separate component (e.g., a short oligonucleotide) that hybridizes to a single-stranded region of the template to be sequenced. Alternatively, the primer and the template strand to be sequenced may each form part of a partially self-complementary nucleic acid strand capable of forming an intra-molecular duplex, such as for example a hairpin loop structure. Hairpin polynucleotides and methods by which they may be attached to solid supports are disclosed in PCT Publication Nos. WO 01/57248 and WO 2005/047301, each of which is incorporated herein by reference. Nucleotides can be added successively to a growing primer, resulting in synthesis of a polynucleotide chain in the 5′ to 3′ direction. The nature of the base which has been added may be determined, particularly but not necessarily after each nucleotide addition, thus providing sequence information for the nucleic acid template. Thus, a nucleotide is incorporated into a nucleic acid strand (or polynucleotide) by joining of the nucleotide to the free 3′ OH group of the nucleic acid strand via formation of a phosphodiester linkage with the 5′ phosphate group of the nucleotide.

The nucleic acid template to be sequenced may be DNA or RNA, or even a hybrid molecule comprised of deoxynucleotides and ribonucleotides. The nucleic acid template may comprise naturally occurring and/or non-naturally occurring nucleotides and natural or non-natural backbone linkages, provided that these do not prevent copying of the template in the sequencing reaction.

In certain embodiments, the target polynucleotides to be sequenced may be attached to a solid support via any suitable linkage method known in the art, for example via covalent attachment. In certain embodiments, target polynucleotides may be attached directly to a solid support (e.g., a silica-based support). However, in other embodiments of the disclosure the surface of the solid support may be modified in some way so as to allow either direct covalent attachment of target polynucleotides, or to immobilize the target polynucleotides through a hydrogel or polyelectrolyte multilayer, which may itself be non-covalently attached to the solid support.

Arrays in which polynucleotides have been directly attached to a support (for example, silica-based supports such as those disclosed in WO 00/06770 (incorporated herein by reference), wherein polynucleotides are immobilized on a glass support by reaction between a pendant epoxide group on the glass with an internal amino group on the polynucleotide. In addition, polynucleotides can be attached to a solid support by reaction of a sulfur-based nucleophile with the solid support, for example, as described in WO 2005/047301 (incorporated herein by reference). A still further example of solid-supported target polynucleotides is where the template polynucleotides are attached to hydrogel supported upon silica-based or other solid supports, for example, as described in WO 00/31148, WO 01/01143, WO 02/12566, WO03/014392, U.S. Pat. No. 6,465,178, and WO 00/53812, each of which is incorporated herein by reference.

A particular surface to which template polynucleotides may be immobilized is a polyacrylamide hydrogel. Polyacrylamide hydrogels are described in the references cited above and in WO2005/065814, which is incorporated herein by reference. Specific hydrogels that may be used include those described in WO 2005/065814 and U.S. Pub. No. 2014/0079923. In one embodiment, the hydrogel is PAZAM (poly(N-(5-azidoacetamidylpentyl) acrylamide-co-acrylamide)).

DNA template molecules can be attached to beads or microparticles, for example, as described in U.S. Pat. No. 6,172,218 (which is incorporated herein by reference). Attachment to beads or microparticles can be useful for sequencing applications. Bead libraries can be prepared where each bead contains different DNA sequences. Exemplary libraries and methods for their creation are described in Nature, 437, 376-380 (2005); Science, 309, 5741, 1728-1732 (2005), each of which is incorporated herein by reference. Sequencing of arrays of such beads using nucleotides set forth herein is within the scope of the disclosure.

Template(s) that are to be sequenced may form part of an “array” on a solid support, in which case the array may take any convenient form. Thus, the method of the disclosure is applicable to all types of high-density arrays, including single-molecule arrays, clustered arrays, and bead arrays. Nucleotides labeled with dye compounds of the present disclosure may be used for sequencing templates on essentially any type of array, including but not limited to those formed by immobilization of nucleic acid molecules on a solid support.

However, nucleotides labeled with dye compounds of the disclosure are particularly advantageous in the context of sequencing of clustered arrays. In clustered arrays, distinct regions on the array (often referred to as sites, or features) comprise multiple polynucleotide template molecules. Generally, the multiple polynucleotide molecules are not individually resolvable by optical means and are instead detected as an ensemble. Depending on how the array is formed, each site on the array may comprise multiple copies of one individual polynucleotide molecule (e.g., the site is homogenous for a particular single- or double-stranded nucleic acid species) or even multiple copies of a small number of different polynucleotide molecules (e.g., multiple copies of two different nucleic acid species). Clustered arrays of nucleic acid molecules may be produced using techniques generally known in the art. By way of example, WO 98/44151 and WO 00/18957, each of which is incorporated herein, describe methods of amplification of nucleic acids wherein both the template and amplification products remain immobilized on a solid support in order to form arrays comprised of clusters or “colonies” of immobilized nucleic acid molecules. The nucleic acid molecules present on the clustered arrays prepared according to these methods are suitable templates for sequencing using nucleotides labeled with dye compounds of the disclosure.

Nucleotides labeled with dye compounds of the present disclosure are also useful in sequencing of templates on single molecule arrays. The term “single molecule array” or “SMA” as used herein refers to a population of polynucleotide molecules, distributed (or arrayed) over a solid support, wherein the spacing of any individual polynucleotide from all others of the population is such that it is possible to individually resolve the individual polynucleotide molecules. The target nucleic acid molecules immobilized onto the surface of the solid support can thus be capable of being resolved by optical means in some embodiments. This means that one or more distinct signals, each representing one polynucleotide, will occur within the resolvable area of the particular imaging device used.

Single molecule detection may be achieved wherein the spacing between adjacent polynucleotide molecules on an array is at least 100 nm, more particularly at least 250 nm, still more particularly at least 300 nm, even more particularly at least 350 nm. Thus, each molecule is individually resolvable and detectable as a single molecule fluorescent point, and fluorescence from said single molecule fluorescent point also exhibits single step photobleaching.

The terms “individually resolved” and “individual resolution” are used herein to specify that, when visualized, it is possible to distinguish one molecule on the array from its neighboring molecules. Separation between individual molecules on the array will be determined, in part, by the particular technique used to resolve the individual molecules. The general features of single molecule arrays will be understood by reference to published applications WO 00/06770 and WO 01/57248, each of which is incorporated herein by reference. Although one use of the labeled nucleotides of the disclosure is in sequencing-by-synthesis reactions, the utility of such nucleotides is not limited to such methods. In fact, the labeled nucleotides described herein may be used advantageously in any sequencing methodology which requires detection of fluorescent labels attached to nucleotides incorporated into a polynucleotide.

In particular, nucleotide conjugates labeled with dye compounds of the disclosure may be used in automated fluorescent sequencing protocols, particularly fluorescent dye-terminator cycle sequencing based on the chain termination sequencing method of Sanger and co-workers. Such methods generally use enzymes and cycle sequencing to incorporate fluorescently labeled dideoxynucleotides in a primer extension sequencing reaction. So-called Sanger sequencing methods, and related protocols (Sanger-type), utilize randomized chain termination with labeled dideoxynucleotides.

### Kits

Some aspects of the present disclosure relate kits for the sequencing method described herein. In particular, a kit may comprise: a first type of nucleotide conjugate comprising a first fluorescent moiety (i.e., a first label); a second type of nucleotide conjugate comprising a second fluorescent moiety (i.e., a second label); and a third type of nucleotide conjugate comprising a third fluorescent moiety (i.e., a third label); wherein each of the first label, the second label, and the third label is spectrally distinct from one another, the first label and the third label are excitable by a first light source, the second label and the third label are excitable by a second light source; wherein the first light source and the second light source have different excitation wavelength; and wherein each of the first label, the second label and the third label has an emission spectrum that is detectable in a single detection channel. In further embodiment, the kit further comprises a fourth type of nucleotide conjugate. In some such embodiment, the fourth type of nucleotide conjugate is unlabeled. The four different types of nucleotide conjugates are A, C, G, and T or U, or non-natural nucleotide analogs thereof. In further embodiments, the four different types of nucleotide conjugates are dATP, dCTP, dGTP and dTTP or dUTP, or non-natural nucleotide analogs thereof. In some embodiments, the first excitation light source has a wavelength from about 490 nm to about 550 nm, from about 510 to about 540 nm, or from about 520 to about 530 nm (e.g., 520 nm). The second light source has an excitation wavelength from about 400 nm to about 480 nm, from about 420 nm to about 470 nm, or from 450 nm to about 460 nm (e.g., 450 nm). In alternative embodiments, the first light source has an excitation wavelength from about 400 nm to about 480 nm, from about 420 nm to about 470 nm, or from 450 nm to about 460 nm (e.g., 450 nm). The second excitation light source has a wavelength from about 490 nm to about 550 nm, from about 500 to about 540 nm, or from about 510 to about 530 nm (e.g., 520 nm). In further embodiments, each of the first label, the second label, and the third label has an emission spectrum that is greater than 550 nm or 560 nm can be collected in a single emission filter or channel. In further embodiments, the single emission detection channel has a detection range of above 560 nm and/or less than 700 nm (e.g., about 565 nm to about 690 nm, about 570 nm to about 670 nm, or about 580 nm to about 650 nm).

The compounds, nucleotide conjugates, or kits that are set forth herein may be used to detect, measure, or identify a biological system (including, for example, processes or components thereof). Exemplary techniques that can employ the compounds, nucleotides or kits include sequencing, expression analysis, hybridization analysis, genetic analysis, RNA analysis, cellular assay (e.g., cell binding or cell function analysis), or protein assay (e.g., protein binding assay or protein activity assay). The use may be on an automated instrument for carrying out a particular technique, such as an automated sequencing instrument. The sequencing instrument may contain two light sources operating at different wavelengths (i.e., first excitation light source and second excitation light source).

In a particular embodiment, the labeled nucleotide conjugates described herein may be supplied in combination with unlabeled or native nucleotides, or any combination thereof. Combinations of nucleotides may be provided as separate individual components (e.g., one nucleotide type per vessel or tube) or as nucleotide mixtures (e.g., two or more nucleotides mixed in the same vessel or tube). In further embodiment, a nucleotide mixture may contain all four types of nucleotides.

As used herein, the term “spectrally distinct” fluorescent dyes or labels refers to fluorescent dyes that absorb light energy at different wavelengths and/or have different Stokes shift that can be distinguished by a fluorescent detection equipment when two or more such dyes are present in one sample. When two labeled nucleotide conjugates are supplied in kit form, it is a feature of some embodiments that the spectrally distinct fluorescent dyes are excitable at different wavelengths, such as, for example by two different light sources. When four nucleotides labeled with fluorescent dye compounds are supplied in kit form, it is a feature of some embodiments that two of the spectrally distinct fluorescent dyes can both be excited at one wavelength and two spectrally distinct dyes can both be excited at another wavelength. Particular excitation wavelengths for the dyes are between 450-460 nm, 490-500 nm, or 520 nm or above (e.g., 532 nm).

In some embodiments of the kits described herein, the nucleotide conjugate with first label is excitable only by the first light source has an absorbance maximum (Aₘₐₓ) of about 450-460 nm. The nucleotide conjugate with second label that is excitable only by the second light source has an Aₘₐₓ of over 520 nm. The nucleotide conjugate with the third label that is excitable at both the first and the second light source (e.g., 450 nm and 520 nm, or 520 nm and 450 nm) has an Aₘₐₓ of about 480 nm to 510 nm, or about 490 nm to 500 nm. Alternatively, the nucleotide conjugate with first label is excitable only by the first light source has an Aₘₐₓ of over 520 nm. The nucleotide conjugate with second label that is excitable only by the second light source has an Aₘₐₓ of about 450-460 nm. The nucleotide conjugate with the third label that is excitable at both the first and the second light source (e.g., 450 nm and 520 nm, or 520 nm and 450 nm) has an Aₘₐₓ of about 480 nm to 510 nm, or about 490 nm to 500 nm. In further embodiments, the emission maximum (Eₘₐₓ) of the nucleotide conjugate with the third label is greater than 550 nm or greater than 560 nm, and the Stokes shift of such labeled nucleotide conjugate is greater than 60 nm. The Eₘₐₓ of the nucleotide conjugate with the first label is greater than 560 nm and Stokes shift of such labeled nucleotide conjugate is greater than 100 nm. The Eₘₐₓ of the nucleotide conjugate with the second label is greater than 560 nm and Stokes shift of such nucleotide conjugate is greater than about 30 nm or greater than about 40 nm. Alternatively, the Eₘₐₓ of the nucleotide conjugate with the third label is greater than 550 nm or greater than 560 nm, and the Stokes shift of such labeled nucleotide conjugate is greater than 60 nm. The Eₘₐₓ of the nucleotide conjugate with the first label is greater than 560 nm and Stokes shift of such nucleotide conjugate is greater than about 30 nm or greater than about 40 nm. The Eₘₐₓ of the nucleotide conjugate with the second label is greater than 560 nm and Stokes shift of such labeled nucleotide conjugate is greater than 100 nm.

In one example, the first light source has an excitation wavelength of 450-460 nm and the second light source has an excitation wavelength of 520 nm. In another example, the first light source has an excitation wavelength of 520 nm and the second light source has an excitation wavelength of 450-460 nm. In some such embodiments, the first type of nucleotide conjugate is C nucleotide (dCTP), the second type of nucleotide conjugate is T nucleotide (dTTP), the third type of nucleotide conjugate is A nucleotide (dATP), and the fourth type of nucleotide is unlabeled G nucleotide (dGTP). In some other embodiments, the first type of nucleotide conjugate is T nucleotide (dTTP), the second type of nucleotide conjugate is C nucleotide (dCTP), the third type of nucleotide conjugate is A nucleotide (dATP), and the fourth type of nucleotide is unlabeled G nucleotide (dGTP). In some other embodiments, the first type of nucleotide conjugate is C nucleotide (dCTP), the second type of nucleotide conjugate is A nucleotide (dATP), the third type of nucleotide conjugate is T nucleotide (dTTP), and the fourth type of nucleotide is unlabeled G nucleotide (dGTP). In yet other embodiments, the first type of nucleotide conjugate is A nucleotide (dATP), the second type of nucleotide conjugate is C nucleotide (dCTP), the third type of nucleotide conjugate is T nucleotide (dTTP), and the fourth type of nucleotide is unlabeled G nucleotide (dGTP). In yet other embodiments, the first type of nucleotide conjugate is A nucleotide (dATP), the second type of nucleotide conjugate is T nucleotide (dTTP), the third type of nucleotide conjugate is C nucleotide (dCTP), and the fourth type of nucleotide is unlabeled G nucleotide (dGTP). In yet other embodiments, the first type of nucleotide conjugate is T nucleotide (dTTP), the second type of nucleotide conjugate is A nucleotide (dATP), the third type of nucleotide conjugate is C nucleotide (dCTP), and the fourth type of nucleotide is unlabeled G nucleotide (dGTP). Alternatively, G or dGTP nucleotide may be labeled with a spectrally distinct label described herein and one of the other three nucleotide conjugates may be unlabeled.

Although kits are exemplified herein in regard to configurations having different nucleotides that are labeled with different dye compounds, it will be understood that kits can include 2, 3, 4 or more different nucleotides that have the same dye compound.

In addition to the labeled nucleotides, the kit may comprise together at least one additional component. The further component(s) may be one or more of the components identified in a method set forth herein or in the Examples section below. Some non-limiting examples of components that can be combined into a kit of the present disclosure are set forth below. In some embodiments, the kit further comprises a DNA polymerase (such as a mutant DNA polymerase) and one or more buffer compositions. One buffer composition may comprise antioxidants such as ascorbic acid or sodium ascorbate, which can be used to protect the dye compounds from photo damage during detection. Additional buffer composition may comprise a reagent can may be used to cleave the 3′ blocking group and/or the cleavable linker. For example, a water-soluble phosphines or water-soluble transition metal catalysts formed from a transition metal and at least partially water-soluble ligands, such as a palladium complex. Various components of the kit may be provided in a concentrated form to be diluted prior to use. In such embodiments a suitable dilution buffer may also be included. Again, one or more of the components identified in a method set forth herein can be included in a kit of the present disclosure.

In some embodiments of the kits described herein, the fluorescent dye compound may be covalently attached to a nucleotide via the nucleotide base. In some such embodiments, the labeled nucleotide may have the dye attached to the C5 position of a pyrimidine base or the C7 position of a 7-deaza purine base, optionally through a linker moiety. For example, the nucleobase may be 7-deaza adenine, and the dye is attached to the 7-deaza adenine at the C7 position, optionally through a linker. The nucleobase may be 7-deaza guanine, and the dye is attached to the 7-deaza guanine at the C7 position, optionally through a linker. The nucleobase may be cytosine, and the dye is attached to the cytosine at the C5 position, optionally through a linker. As another example, the nucleobase may be thymine or uracil and the dye is attached to the thymine or uracil at the C5 position, optionally through a linker. In any embodiments of the nucleotide or nucleotide conjugate described herein, the nucleotide or nucleotide conjugate may contain a 3′ hydroxyl blocking group.

### 3′ Hydroxyl Blocking Groups

The labeled nucleotide conjugate used in the incorporation mixture may also have a blocking group covalently attached to the ribose or deoxyribose sugar of the nucleotide. The blocking group may be attached at any position on the ribose or deoxyribose sugar. In particular embodiments, the blocking group is at the 3′ OH position of the ribose or deoxyribose sugar of the nucleotide. Various 3′ OH blocking group are disclosed in WO2004/018497 and WO2014/139596, which are hereby incorporated by references. For example, the blocking group may be azidomethyl (—CH₂N₃) or substituted azidomethyl (e.g., —CH(CHF₂)N₃ or CH(CH₂F)N₃), or allyl connecting to the 3′ oxygen atom of the ribose or deoxyribose moiety. In some embodiments, the 3′ blocking group is azidomethyl, forming 3′-OCH₂N₃ with the 3′ carbon of the ribose or deoxyribose.

In some other embodiments, the 3′ blocking group and the 3′ oxygen atoms form an acetal group of the structure

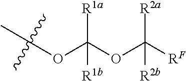

covalent attached to the 3′ carbon of the ribose or deoxyribose, wherein:

each R^{1a} and R^{1b} is independently H, C₁-C₆ alkyl, C₁-C₆ haloalkyl, C₁-C₆ alkoxy, C₁-C₆ haloalkoxy, cyano, halogen, optionally substituted phenyl, or optionally substituted aralkyl;

each R^{2a} and R^{2b} is independently H, C₁-C₆ alkyl, C₁-C₆ haloalkyl, cyano, or halogen;

alternatively, R^{1a} and R^{2a} together with the atoms to which they are attached form an optionally substituted five to eight membered heterocyclyl group;

R^{F} is H, optionally substituted C₂-C₆ alkenyl, optionally substituted C₃-C₇ cycloalkenyl, optionally substituted C₂-C₆ alkynyl, or optionally substituted (C₁-C₆ alkylene)Si(R^{3a})₃; and each R^{1a} is independently H, C₁-C₆ alkyl, or optionally substituted C₆-C₁₀ aryl.

Additional 3′ OH blocking groups are disclosed in U.S. Publication No. 2020/0216891 A1, which is incorporated by reference in its entirety. Non-limiting examples of the acetal blocking group

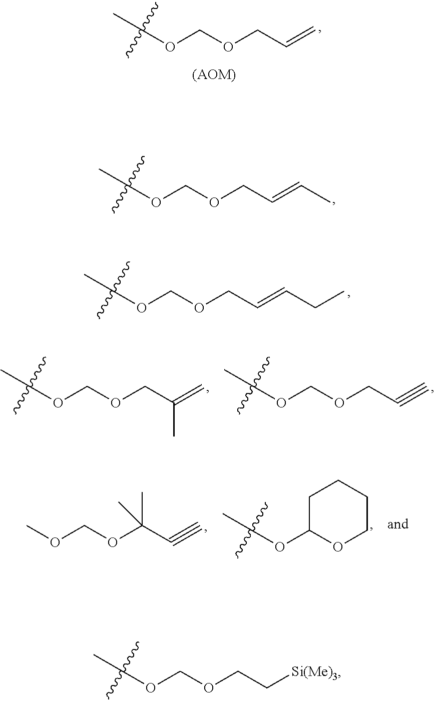

each covalently attached to the 3′ carbon of the ribose or deoxyribose.

### Deprotection of the 3′-OH Blocking Groups

In some embodiments, the azidomethyl 3′ hydroxy protecting group may be removed or deprotected by using a water-soluble phosphine reagent. Non-limiting examples include tris(hydroxymethyl)phosphine (THMP), tris(hydroxyethyl)phosphine (THEP) or tris(hydroxylpropyl)phosphine (THP or THPP). 3′-acetal blocking groups described herein may be removed or cleaved under various chemical conditions. For acetal blocking groups

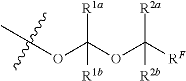

that contain a vinyl or alkenyl moiety, non-limiting cleaving condition includes a Pd(II) complex, such as Pd(OAc)₂ or allylPd(II) chloride dimer, in the presence of a phosphine ligand, for example tris(hydroxymethyl)phosphine (THMP), or tris(hydroxylpropyl)phosphine (THP or THPP). For those blocking groups containing an alkynyl group (e.g., an ethynyl), they may also be removed by a Pd(II) complex (e.g., Pd(OAc)₂ or allyl Pd(II) chloride dimer) in the presence of a phosphine ligand (e.g., THP or THMP).

Palladium Cleavage Reagents

In some embodiments, the 3′ hydroxyl blocking group described herein may be cleaved by a palladium catalyst. In some such embodiments, the Pd catalyst is water soluble. In some such embodiments, is a Pd(0) complex (e.g., Tris(3,3′,3″-phosphinidynetris (benzenesulfonato) palladium(0) nonasodium salt nonahydrate). In some instances, the Pd(0) complex may be generated in situ from reduction of a Pd (II) complex by reagents such as alkenes, alcohols, amines, phosphines, or metal hydrides. Suitable palladium sources include Na₂PdCl₄, Pd(CH₃CN)₂Cl₂, (PdCl(C₃H₅))₂, [Pd(C₃H₅)(THP)]Cl, [Pd(C₃H₅)(THP)₂]Cl, Pd(OAc)₂, Pd(Ph₃)₄, Pd(dba)₂, Pd(Acac)₂, PdCl₂(COD), and Pd(TFA)₂. In one such embodiment, the Pd(0) complex is generated in situ from Na₂PdCl₄. In another embodiment, the palladium source is allyl palladium (II) chloride dimer [(PdCl(C₃H₅))₂]. In some embodiments, the Pd(0) complex is generated in an aqueous solution by mixing a Pd (II) complex with a phosphine. Suitable phosphines include water soluble phosphines, such as tris(hydroxypropyl)phosphine (THP), tris(hydroxymethyl)phosphine (THMP), 1,3,5-triaza-7-phosphaadamantane (PTA), bis(p-sulfonatophenyl)phenylphosphine dihydrate potassium salt, tris(carboxyethyl)phosphine (TCEP), and triphenylphosphine-3,3′,3″-trisulfonic acid trisodium salt.

In some embodiments, the Pd(0) is prepared by mixing a Pd(II) complex [(PdCl(C₃H₅))₂] with THP in situ. The molar ratio of the Pd(II) complex and the THP may be about 1:2, 1:3, 1:4, 1:5, 1:6, 1:7, 1:8, 1:9, or 1:10. In some further embodiments, one or more reducing agents may be added, such as ascorbic acid or a salt thereof (e.g., sodium ascorbate). In some embodiments, the cleavage mixture may contain additional buffer reagents, such as a primary amine, a secondary amine, a tertiary amine, a carbonate salt, a phosphate salt, or a borate salt, or combinations thereof. In some further embodiments, the buffer reagent comprises ethanolamine (EA), tris(hydroxymethyl)aminomethane (Tris), glycine, sodium carbonate, sodium phosphate, sodium borate, 2-dimethylethanolamine (DMEA), 2-diethylethanolamine (DEEA), N,N,N′,N′-tetramethylethylenediamine(TEMED), or N,N,N′,N′-tetraethylethylenediamine (TEEDA), or combinations thereof. In one embodiment, the buffer reagent is DEEA. In another embodiment, the buffer reagent contains one or more inorganic salts such as a carbonate salt, a phosphate salt, or a borate salt, or combinations thereof. In one embodiment, the inorganic salt is a sodium salt.

Linkers

The fluorescent labels may be covalently attached to a nucleotide via a cleavable linker. Use of the term “cleavable linker” is not meant to imply that the whole linker is required to be removed. The cleavage site can be located at a position on the linker that ensures that part of the linker remains attached to the dye and/or substrate moiety after cleavage. Cleavable linkers may be, by way of non-limiting example, electrophilically cleavable linkers, nucleophilically cleavable linkers, photocleavable linkers, cleavable under reductive conditions (for example disulfide or azide containing linkers), oxidative conditions, cleavable via use of safety-catch linkers and cleavable by elimination mechanisms. The use of a cleavable linker to attach the dye compound to a substrate moiety ensures that the label can, if required, be removed after detection, avoiding any interfering signal in downstream steps.

Useful linker groups may be found in PCT Publication No. WO 2004/018493 (herein incorporated by reference), examples of which include linkers that may be cleaved using water-soluble phosphines or water-soluble transition metal catalysts formed from a transition metal and at least partially water-soluble ligands. In aqueous solution the latter form at least partially water-soluble transition metal complexes. Such cleavable linkers can be used to connect bases of nucleotides to labels such as the dyes set forth herein.

Particular linkers include those disclosed in PCT Publication No. WO 2004/018493 (herein incorporated by reference) such as those that include moieties of the formulae:

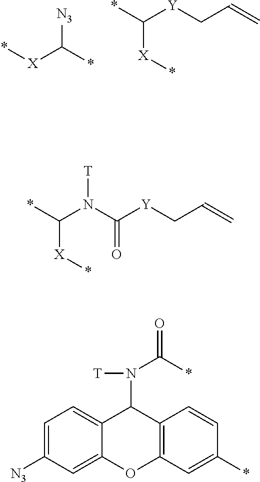

(wherein X is selected from the group comprising O, S, NH and NQ wherein Q is a C1-10 substituted or unsubstituted alkyl group, Y is selected from the group comprising O, S, NH and N(allyl), T is hydrogen or a C₁-C₁₀ substituted or unsubstituted alkyl group and * indicates where the moiety is connected to the remainder of the nucleotide or nucleoside). In some aspect, the linkers connect the bases of nucleotides to labels such as, for example, the dye compounds described herein.

Additional examples of linkers include those disclosed in U.S. Publication No. 2016/0040225 (herein incorporated by reference), such as those include moieties of the formulae:

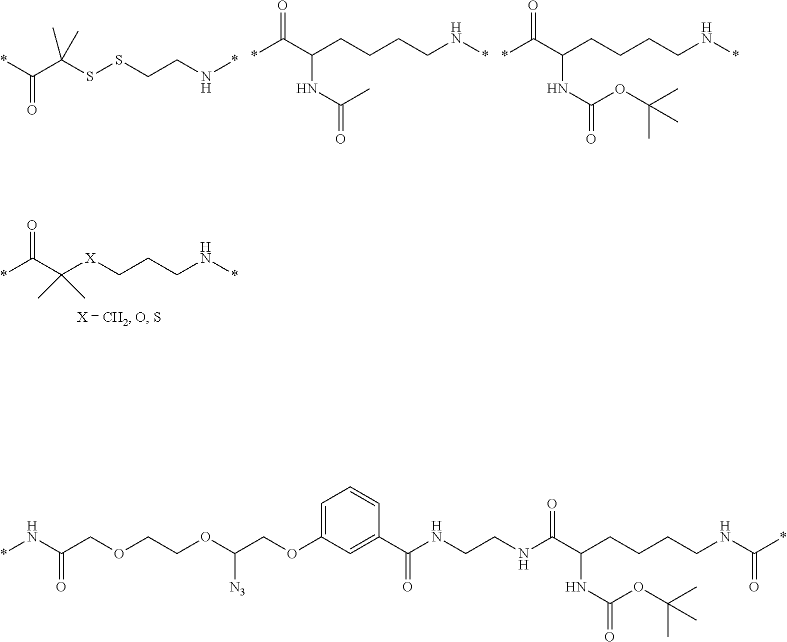

(wherein * indicates where the moiety is connected to the remainder of the nucleotide or nucleoside). The linker moieties illustrated herein may comprise the whole or partial linker structure between the nucleotides/nucleosides and the labels. The linker moieties illustrated herein may comprise the whole or partial linker structure between the nucleotides/nucleosides and the labels.

Additional examples of linkers include moieties of the formula:

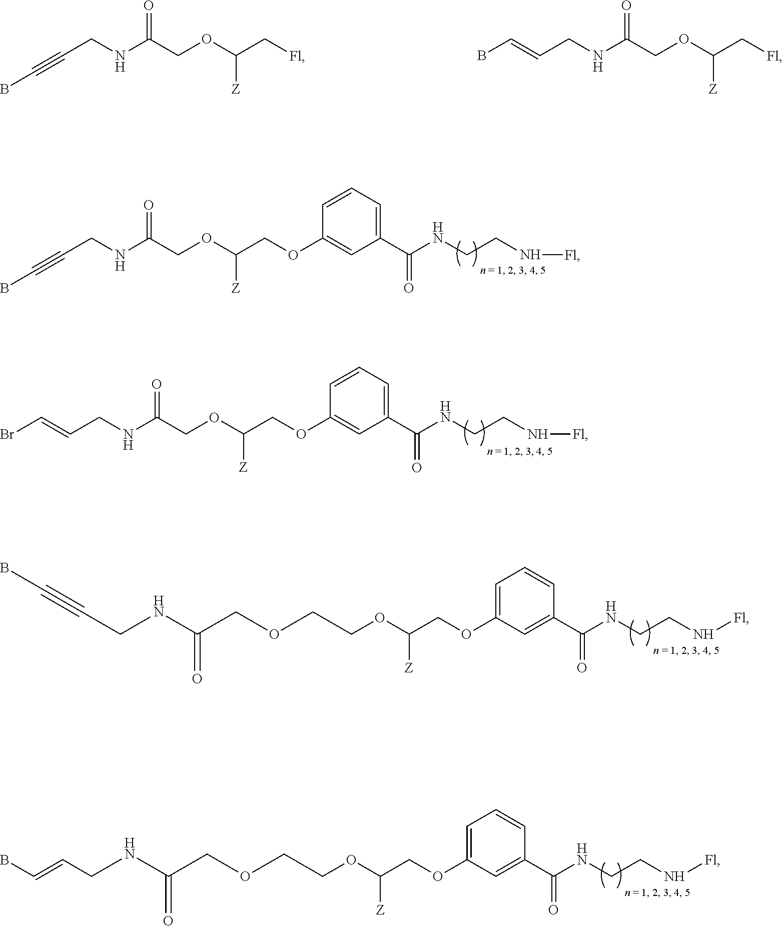

wherein B is a nucleobase; Z is N₃ (azido), —O—C₁-C₆ alkyl, —O—C₂-C₆ alkenyl, or —O—C₂-C₆ alkynyl; and F1 comprises a dye moiety, which may contain additional linker structure. One of ordinary skill in the art understands that the dye compound described herein is covalently bounded to the linker by reacting a functional group of the dye compound (e.g., carboxyl) with a functional group of the linker (e.g., amino). In one embodiment, the cleavable linker comprises

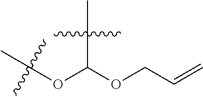 [SMILES: *OC(*)OCC=C]

(“AOL” linker moiety) where Z is —O-allyl. Further embodiments of the cleavable linker are disclosed in U.S. application Ser. No. 17/353,512, which is hereby incorporated by reference in its entirety.

In particular embodiments, the length of the linker between a fluorescent dye (fluorophore) and a guanine base can be altered, for example, by introducing a polyethylene glycol spacer group, thereby increasing the fluorescence intensity compared to the same fluorophore attached to the guanine base through other linkages known in the art. Exemplary linkers and their properties are set forth in PCT Publication No. WO 2007/020457 (herein incorporated by reference). The design of linkers, and especially their increased length, can allow improvements in the brightness of fluorophores attached to the guanine bases of guanosine nucleotides when incorporated into polynucleotides such as DNA. Thus, when the dye is for use in any method of analysis which requires detection of a fluorescent dye label attached to a guanine-containing nucleotide, it is advantageous if the linker comprises a spacer group of formula —((CH₂)₂O)ₙ—, wherein n is an integer between 2 and 50, as described in WO 2007/020457.

A dye may be attached to any position on the nucleotide base, for example, through a linker. In particular embodiments, Watson-Crick base pairing can still be carried out for the resulting analog. Particular nucleobase labeling sites include the C5 position of a pyrimidine base or the C7 position of a 7-deaza purine base. As described above a linker group may be used to covalently attach a dye to the nucleoside or nucleotide.

Nucleotides labeled with the dyes described herein may have the formula:

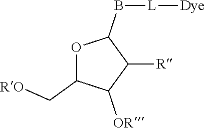

where Dye is a dye compound (label) moiety described herein (after covalent bonding between a functional group of the dye and a functional group of the linker “L”); B is a nucleobase, such as, for example uracil, thymine, cytosine, adenine, 7-deaza adenine, guanine, 7-deaza guanine, and the like; L is an optional linker which may or may not be present; R′ can be H, or —OR′ is monophosphate, diphosphate, triphosphate, thiophosphate, a phosphate ester analog, —O— attached to a reactive phosphorous containing group, or —O— protected by a blocking group; R″ is H or OH; and R′″ is H, a 3′ OH blocking group described herein, or —OR′″ forms a phosphoramidite. Where —OR′″ is phosphoramidite, R′ is an acid-cleavable hydroxyl protecting group which allows subsequent monomer coupling under automated synthesis conditions. In some further embodiments, B comprises

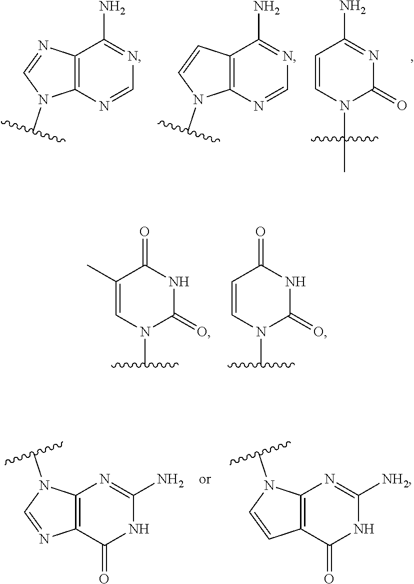

or optionally substituted derivatives and analogs thereof. In some further embodiments, the labeled nucleobase comprises the structure

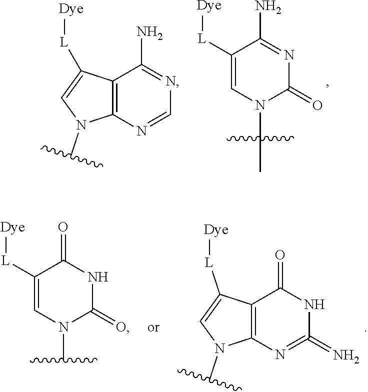

In a particular embodiment, the blocking group is separate and independent of the dye compound, i.e., not attached to it. Alternatively, the dye may comprise all or part of the 3′ OH blocking group. Thus R′″ can be a 3′ OH blocking group which may or may not comprise the dye compound.

In yet another alternative embodiment, there is no blocking group on the 3′ carbon of the pentose sugar and the dye (or dye and linker construct) attached to the base, for example, can be of a size or structure sufficient to act as a block to the incorporation of a further nucleotide. Thus, the block can be due to steric hindrance or can be due to a combination of size, charge and structure, whether or not the dye is attached to the 3′ position of the sugar.

In still yet another alternative embodiment, the blocking group is present on the 2′ or 4′ carbon of the pentose sugar and can be of a size or structure sufficient to act as a block to the incorporation of a further nucleotide.

The use of a blocking group allows polymerization to be controlled, such as by stopping extension when a labeled nucleotide is incorporated. If the blocking effect is reversible, for example, by way of non-limiting example by changing chemical conditions or by removal of a chemical block, extension can be stopped at certain points and then allowed to continue.

In a particular embodiment, the linker (between dye and nucleotide) and blocking group are both present and are separate moieties. In particular embodiments, the linker and blocking group are both cleavable under the same or substantially similar conditions. Thus, deprotection and deblocking processes may be more efficient because only a single treatment will be required to remove both the dye compound and the blocking group. However, in some embodiments a linker and blocking group need not be cleavable under similar conditions, instead being individually cleavable under distinct conditions.

The disclosure also encompasses polynucleotides incorporating dye compounds. Such polynucleotides may be DNA or RNA comprised respectively of deoxyribonucleotides or ribonucleotides joined in phosphodiester linkage. Polynucleotides may comprise naturally occurring nucleotides, non-naturally occurring (or modified) nucleotides other than the labeled nucleotides described herein or any combination thereof, in combination with at least one modified nucleotide (e.g., labeled with a dye compound) as set forth herein. Polynucleotides according to the disclosure may also include non-natural backbone linkages and/or non-nucleotide chemical modifications. Chimeric structures comprised of mixtures of ribonucleotides and deoxyribonucleotides comprising at least one labeled nucleotide are also contemplated.

Non-limiting exemplary labeled nucleotide conjugates as described herein include:

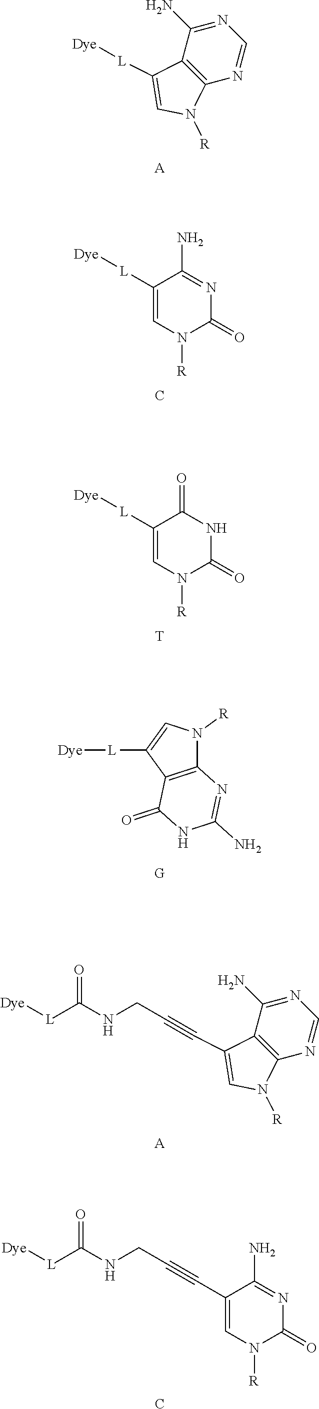

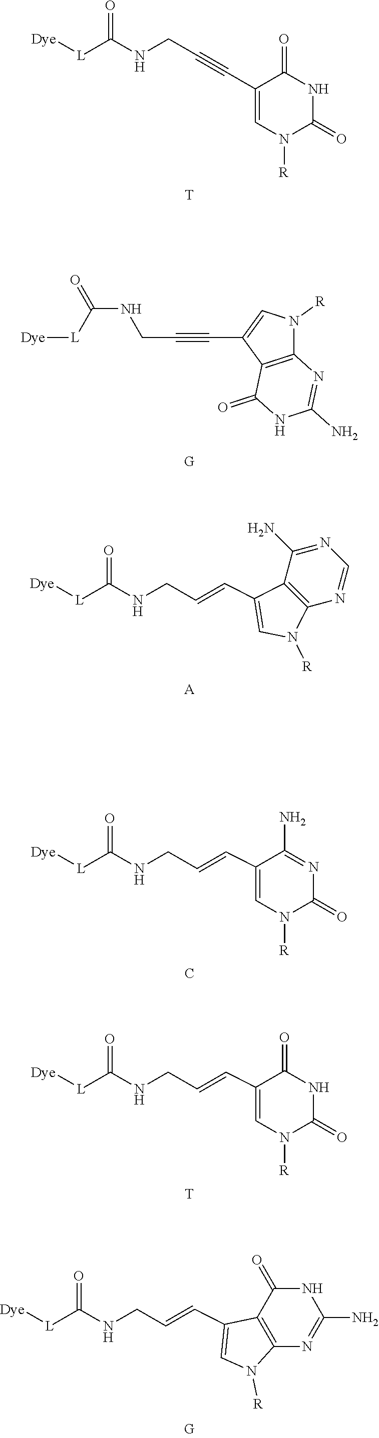

wherein L represents a linker and R represents a ribose or deoxyribose moiety as described above, or a ribose or deoxyribose moiety with the 5′ position substituted with mono-, di- or tri-phosphates.

In some embodiments, non-limiting exemplary fully functionalized nucleotide conjugates including a cleavable linker and a fluorescent moiety are shown below:

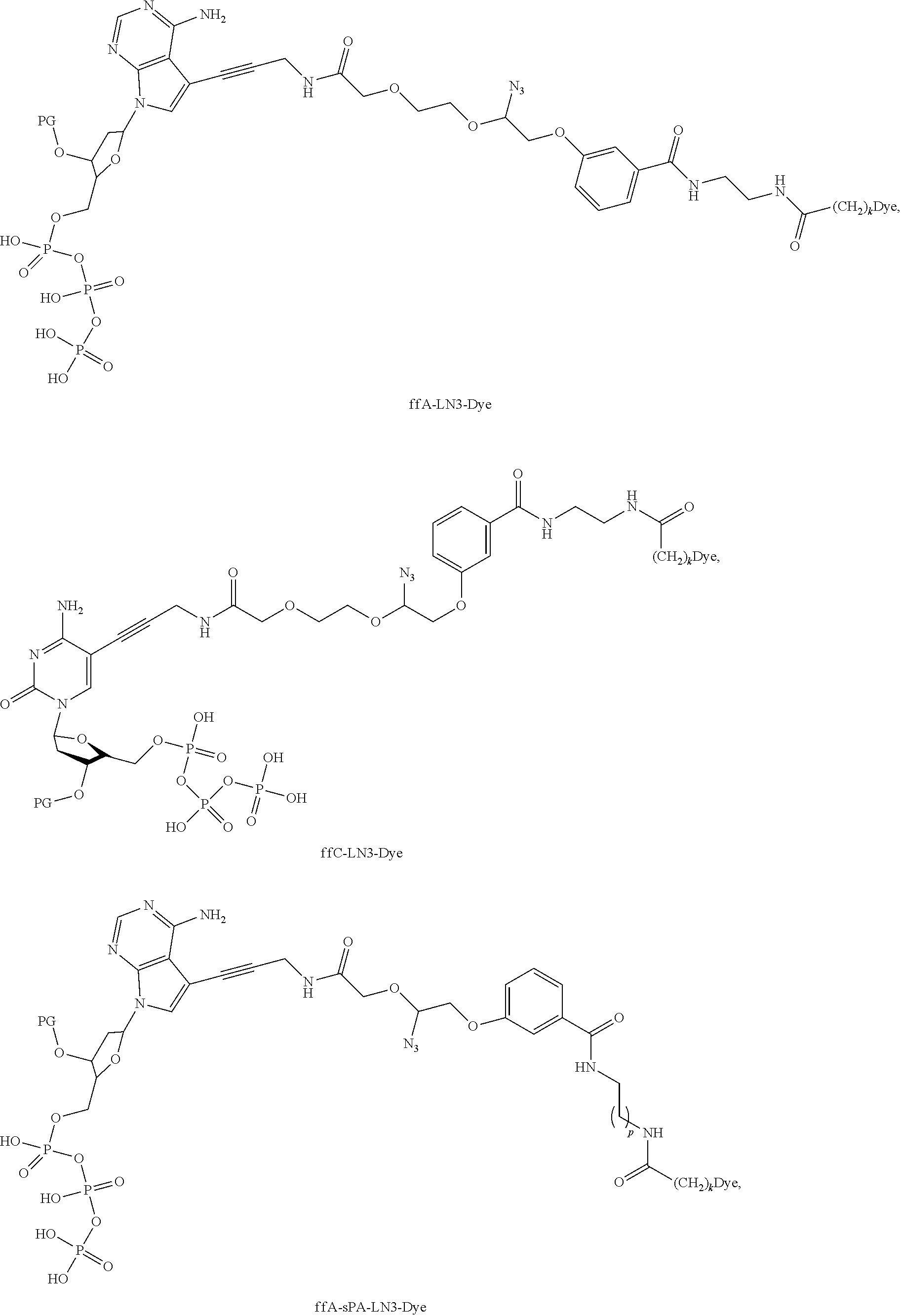

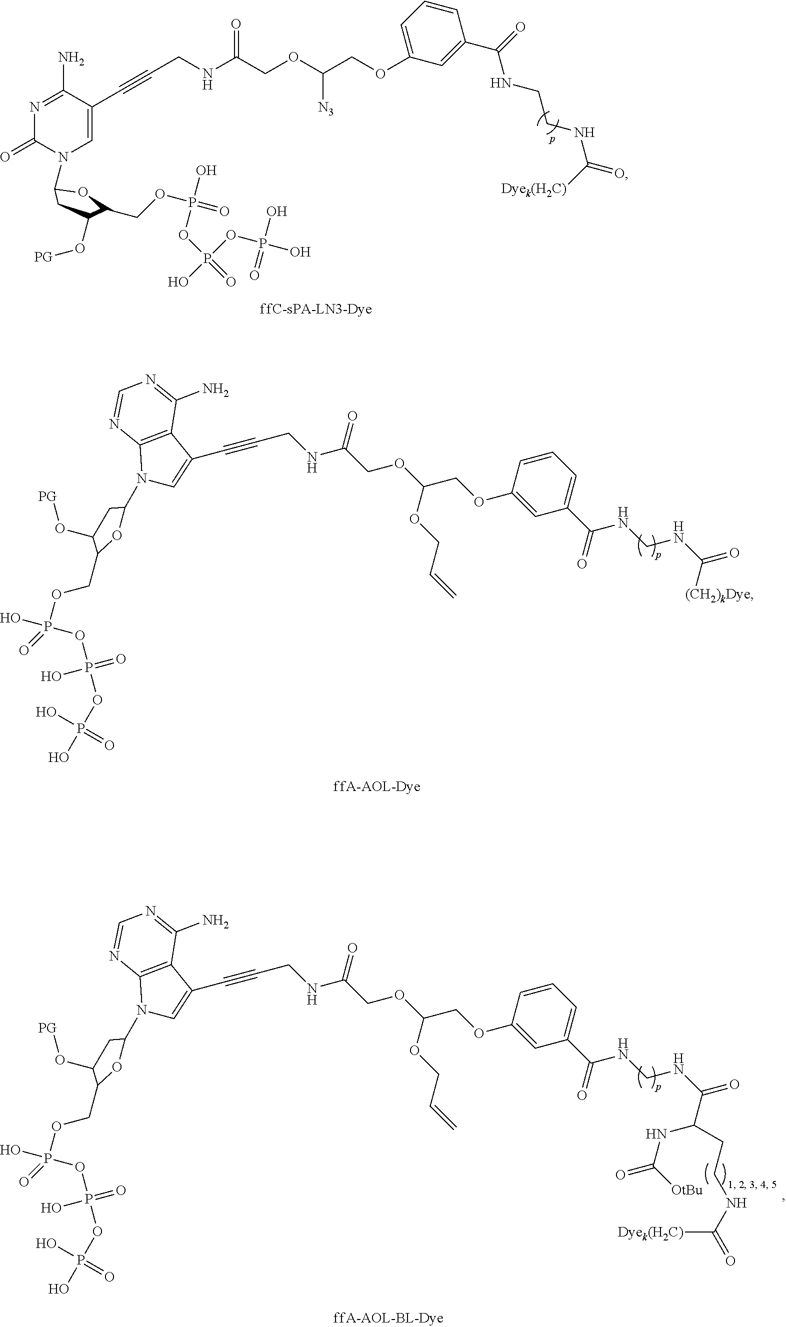

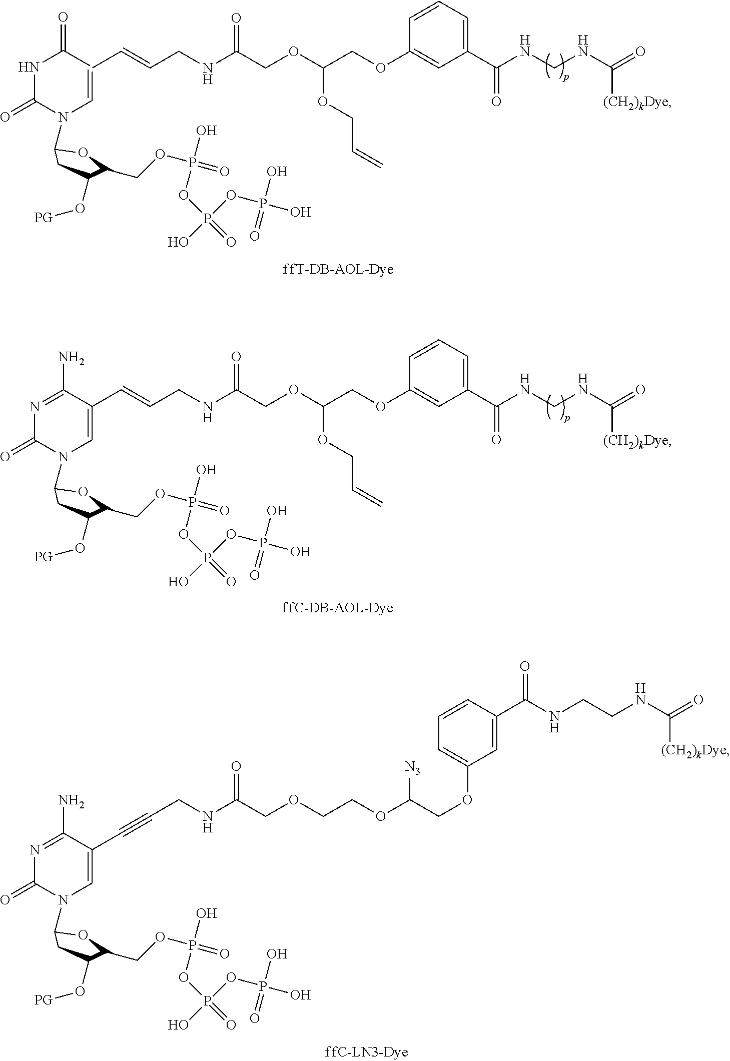

wherein PG stands for the 3′ OH blocking groups described herein; p is an integer of 1, 2, 3, 4, 5, 6, 7, 8, 9, or 10; and k is 0, 1, 2, 3, 4, or 5. In one embodiment, —O-PG is AOM. In another embodiment, —O—PG is —O-azidomethyl. In one embodiment, k is 5. In some further embodiments, p is 1, 2 or 3; and k is 5.

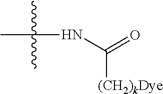

refers to the connection point of the Dye with the cleavable linker as a result of a reaction between an amino group of the linker moiety and the carboxyl group of the Dye. In any embodiments of the labeled nucleotide described herein, the nucleotide is a nucleotide triphosphate.

### EXAMPLES

Additional embodiments are disclosed in further detail in the following examples, which are not in any way intended to limit the scope of the claims.

### Example 1. Sequencing Experiments on Illumina iSeq™ 100 Instrument

In this example, the two-channel excitation and one-channel detection sequencing method described herein was used on an Illumina iSeq™ 100 instrument, which had been set up to take the first image with a green excitation light (˜520 nm) and the second image with a blue excitation light (˜450 nm). The sequencing recipe was modified in order to perform a standard SBS cycle (incorporation, followed by imaging, followed by cleavage) for 1×300 cycles. The incorporation mixture comprises the following four ffNs: an ffA labeled with a chromenoquinoline dye A that is excitable with both the green light at 520 nm and the blue light at 450 nm, an ffC labeled with a coumarin dye B that is excitable with the blue light at 450 nm, an ffT labeled with a polymethine dye C that is excitable with the green light and an unlabeled ffG (dark dGTP) in 50 mM ethanolamine buffer, pH 9.6, 50 mM NaCl, 1 mM EDTA, 0.2% CHAPS, 4 mM MgSO₄ and a DNA polymerase. The structures of the ffC, ffA, ffT and ffG are illustrated below:

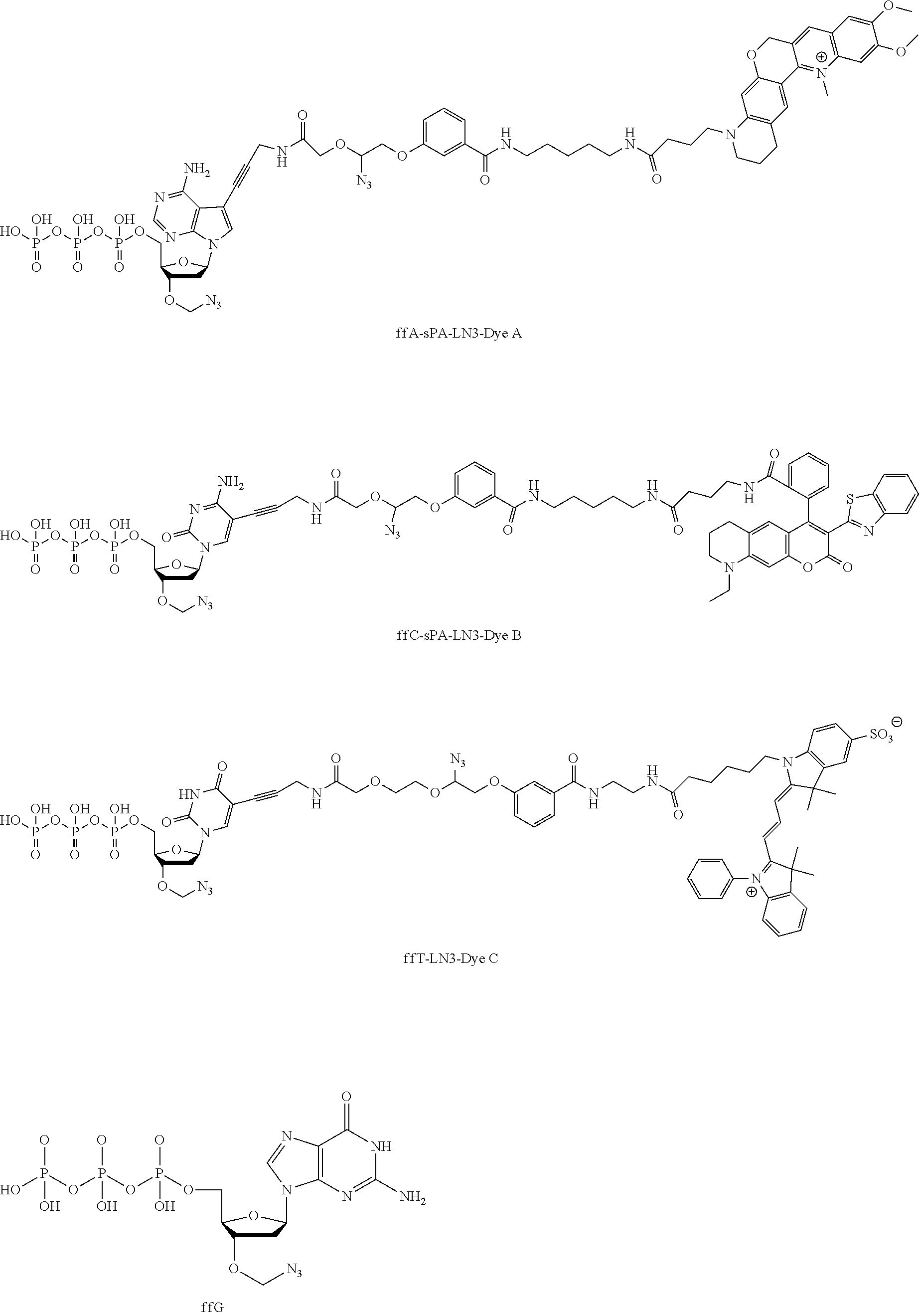

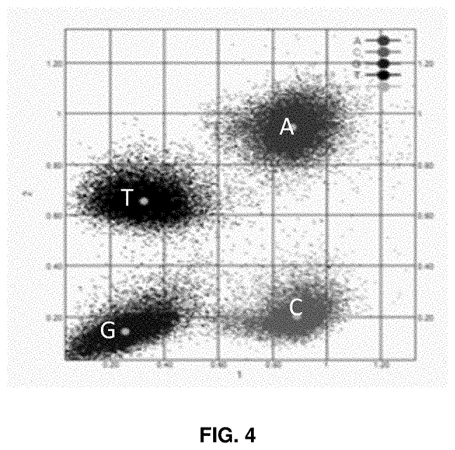

FIG. 4 shows the scatterplot obtained for the incorporation mix at cycle 26. The scatterplot indicated good separations of the clouds and the usability of the ffN set.

Next, the same ffN sets were used in a 2×300 cycles run on an Illumina iSeq™ 100 instrument. The instrument was set up to take the first image with a green excitation light and the second image with the blue excitation light, and the recipe was modified in order to perform a standard SBS cycle (incorporation, followed by imaging, followed by cleavage) for 2×300 cycles. The sequencing metrics are summarized in Table 1 below. It was observed that the phasing and prephasing values were improved compared to those obtained with standard iSeq™ 100 reagents. In addition, with this set of ffNs the sequencing recipe uses only half the volume of the wash buffer and scanning buffer compared to the standard iSeq™ 100 recipe. These features allowed to achieve an extended read length of 2×300 cycles with low error rates and high % Q30 values and a total run time of approximately 28 hours.

TABLE 1
iSeq™ 100 Sequencing Metrics (2 × 300 cycles)

| | | | Error Rate | | % Q30 (Last 18 |
|---|---|---|---|---|---|
| | Phasing | Prephasing | (%) | % Q30 | Cycles) |
| Read | 0.111 | 0.129 | 0.81 | 91.49 | 72.75 |
| Read | 0.11 | 0.141 | 1.01 | 85.57 | 55.58 |

### Example 2. Sequencing Experiments on Illumina iSeq™ 100 Instrument

In this example, the two-channel excitation and one-channel detection sequencing method described herein was used on an Illumina iSeq™ 100 instrument, which had been set up to take the first image with a green excitation light (˜520 nm) and the second image with a blue excitation light (˜450 nm). The standard sequencing recipe was used to perform the SBS cycle (incorporation, followed by imaging, followed by cleavage) for 2×300 cycles. The incorporation mixture comprises the following four ffNs: an ffA labeled with a chromenoquinoline dye A that is excitable with both the green light at 520 nm and the blue light at 450 nm, an ffC labeled with a coumarin dye B that is excitable with the blue light at 450 nm, an ffT labeled with a polymethine dye C that is excitable with the green light and an unlabeled ffG (dark dGTP) in 50 mM glycine buffer pH 9.8, 50 mM NaCl, 1 mM EDTA, 0.2% CHAPS, 4 mM MgSO₄ and a DNA polymerase. The cleavage solution used in these experiments contained 100 mM diethylethanolamine buffer pH 9.5, 100 mM tris(hydroxypropyl)phosphine, 10 mM [AllylPdCl]₂, 10 mM sodium ascorbate, 1 M NaCl, 0.1% Tween20. The structures of the ffC, ffA, ffT and ffG are illustrated below:

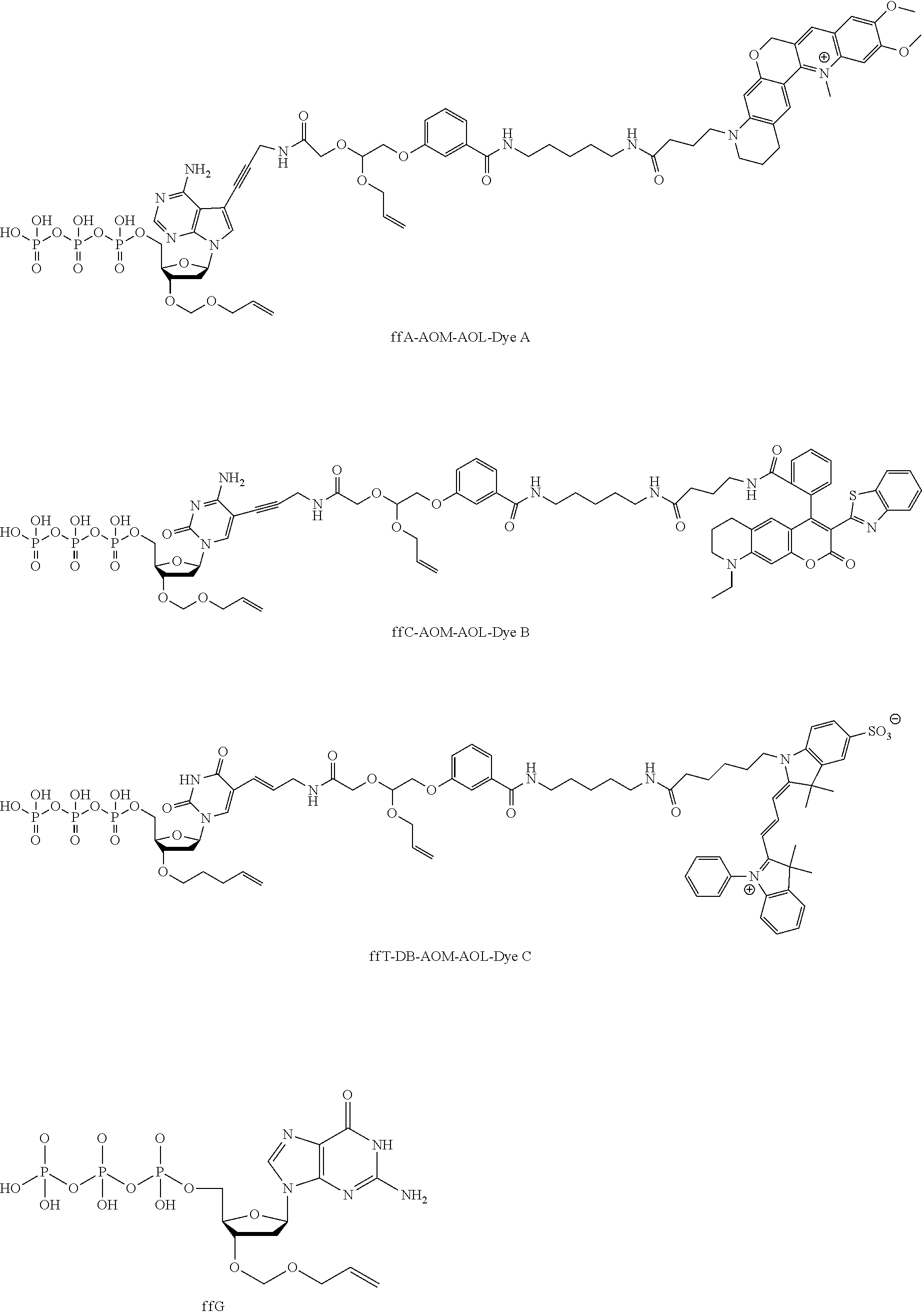

Table 2 shows the cycle time, phasing, prephasing, PhiX error rates, and % Q30 metrics of a 2×150 cycles run and a 2×300 cycles run on an Illumina iSeq™ 100 instrument using the ffN set described in this example. It was observed that using this set of ffNs, it was possible to reduce the cycle time of a 2×150 run to 60 seconds, an approximately 2-fold reduction compared to the standard iSeq™ cartridge and recipe (approximately 141 seconds), while retaining good sequencing metrics. In addition, the sequencing recipe with this set of ffNs uses only half the volume of the wash buffer and scanning buffer compared to the standard iSeq™ 100 recipe (1-Ex chemistry). These features allowed to achieve an extended read length of 2×300 cycles with low error rates and high % Q30 values, using a cycle time comparable to the one used in the standard iSeq™ 100 recipe with the standard iSeq™ cartridge. The total time of a full 2×300 cycles run with this set of ffNs on iSeq™ 100 was around 28 hours, which is a 2-fold reduction compared to a 2×300 cycles run performed on an Illumina MiSeq® using standard reagents and recipes (approximately 56 hours).

TABLE 2
iSeq™ 100 Sequencing Metrics (2 × 150 and 2 × 300 cycles)

| | N | Cycle | | | Error | |
|---|---|---|---|---|---|---|
| | of | time | | | Rate | % |
| | cycles | (s) | Phasing | Prephasing | (%) | Q30 |
| Read 1 | 150 | 60 | 0.156 | 0.059 | 0.53 | 92.84 |
| Read 2 | 150 | 60 | 0.197 | 0.07 | 0.53 | 91.16 |
| Read 1 | 300 | 138 | 0.128 | 0.064 | 0.74 | 93.05 |
| Read 2 | 300 | 138 | 0.142 | 0.053 | 0.65 | 90.9 |

Table 3 shows the cycle time, phasing, prephasing, PhiX error rates, and % Q30 metrics of a 2×150 cycles run on an Illumina iSeq™ 100 instrument using the ffN set described in this example, where the flowcell pixel size has been reduced from 1.75 μm to 1 μm. The change of flowcell pixel size results in smaller pitch and a shorter lightpipe for better signal but also has the potential concerns of low signal, high background noise and high crosstalk. It was observed that the sequencing metrics had overall very good quality on the 1 μm flowcell, and were comparable to those obtained from the 1.75 μm flowcell.

TABLE 3
iSeq™ 100 Sequencing Metrics (2 × 150 cycles) on 1 μm flowcell

| Read | Cycle time | Phasing | Prephasing | % Q30 | Error Rate (%) |
|---|---|---|---|---|---|
| Read 1 | 31 s | 0.178 | 0.082 | 96.61 | 0.33 ± 0.52 |
| Read 2 | | 0.273 | 0.041 | 93.73 | 0.67 ± 0.92 |

1. A method for determining the sequence of a target polynucleotide, comprising:
(a) contacting a primer polynucleotide with a mixture comprising one or more of four different types of nucleotide conjugates, wherein a first type of nucleotide conjugate comprises a first label, a second type of nucleotide conjugate comprises a second label, and a third type of nucleotide conjugate comprises a third label, wherein each of the first label, the second label, and the third label is spectrally distinct from one another, and wherein the primer polynucleotide is complementary to at least a portion of the target polynucleotide;
(b) incorporating a nucleotide conjugate from the mixture in the primer polynucleotide to produce an extended primer polynucleotide;
(c) performing a first imaging event using a first excitation light source and collecting a first emission signal from the extended primer polynucleotide;
(d) performing a second imaging event using a second excitation light source and collecting a second emission signal from the extended primer polynucleotide;
wherein the first excitation light source and the second excitation light source have different wavelengths; and wherein first emission signal and the second emission signal are collected in a single emission detection channel.

2. The method of claim 1, wherein the fourth type of nucleotide conjugate is not labeled or is labeled with a fluorescent moiety that does not emit a detectable signal from either the first imaging event or the second imaging event.

3. The method of claim 1, wherein the incorporation of the first type of the nucleotide conjugate is determined by a signal state in the first imaging event and a dark state in the second imaging event.

4. The method of claim 1, wherein the incorporation of the second type of the nucleotide conjugates is determined by a dark state in the first imaging event and a signal state in the second imaging event.

5. The method of claim 1, wherein the incorporation of the third type of the nucleotide conjugates is determined by a signal state in both the first imaging event and the second imaging event.

6. The method of claim 1, wherein the incorporation of the fourth type of the nucleotide conjugates is determined by a dark state in both the first imaging event and the second imaging event.

7. The method of claim 1, wherein the four types of nucleotide conjugates comprise dATP, dCTP, dGTP and dTTP or dUTP, or non-natural nucleotide analogs thereof, and each of the four types of nucleotide conjugates in the mixture has a 3′ hydroxyl blocking group.

8. (canceled)

9. The method of claim 1, further comprising: (e) removing the 3′ hydroxyl blocking group and the label from the incorporated nucleotide conjugate after the second imaging event, and prior to the next sequencing cycle.

10. The method of claim 9, further comprising:
repeating steps (a)-(e) for multiple cycles; and
determining the sequence of the target polynucleotide based on the sequentially incorporated nucleotide conjugates.

11. The method of claim 10, wherein steps (a)-(e) are repeated for at least 50 cycles.

12. (canceled)

13. The method of claim 1, wherein the first excitation light source has a wavelength of about 400 nm to about 480 nm, or about 450 nm to about 460 nm.

14. (canceled)

15. The method of claim 1, wherein the second excitation light source has a wavelength of about 490 nm to about 550 nm, or about 510 nm to about 530 nm.

16. (canceled)

17. The method of claim 1, wherein the first excitation light source has a wavelength of about 490 nm to about 550 nm, or about 510 nm to about 530 nm.

18. (canceled)

19. The method of claim 1, wherein the second excitation light source have a wavelength of about 400 nm to about 480 nna or about 450 nm to about 460 nm.

20. (canceled)

21. The method of claim 1, wherein the single emission detection channel has a detection spectrum range above 560 nm.

22. The method of claim 1, wherein the method does not comprise a chemical modification of any nucleotide conjugates in the mixture between the first imaging event and the second imaging event.

23. The method of claim 1, wherein the target polynucleotide is immobilized to a solid support, and wherein the solid support comprises a plurality of immobilized target polynucleotides.

24. (canceled)

25. The method of claim 23, wherein the solid support comprises a patterned flow cell, and wherein the patterned flow cell comprises a plurality of nanowells and comprising the plurality of immobilized target polynucleotides.

26. (canceled)

27. The method of claim 23, wherein the solid support further comprises a complementary metal-oxide-semiconductor (CMOS) chip.

28. A kit for sequencing application, comprising:
a first type of nucleotide conjugate comprises a first label;
a second type of nucleotide conjugate comprises a second label; and
a third type of nucleotide conjugate comprises a third label;
wherein each of the first label, the second label, and the third label is spectrally distinct from one another, the first label and the third label are excitable using a first light source wavelength, the second label and the third label are excitable using a second light source wavelength that is different from the first light source wavelength; and
wherein each of the first label, the second label and the third label has an emission spectrum that is detectable in a single detection channel.

29. The kit of claim 28, further comprising a fourth type of nucleotide, and wherein the fourth type of nucleotide is unlabeled (dark).

30. The kit of claim 28, wherein the first light source has a wavelength of about 400 nm to about 480 nm, or about 450 nm to about 450 nm to about 460 nm, and the second light source has a wavelength of about 490 nm to about 550 nm, or about 510 nm to about 530 nm.

31. (canceled)

32. (canceled)

33. (canceled)

34. The kit of claim 28, wherein the single emission detection channel has a detection spectrum range of greater than about 560 nm.

35. The kit according to of claim 28, further comprising a DNA polymerase and one or more buffer compositions.

36. The method of claim 1, wherein the method is carried out in an array format, wherein multiple target polynucleotides are sequenced in parallel.
